(19) United States
(12) Patent Application Publication
(10) Pub. No.: US 2024/0428922 A1
(43) Pub. Date: Dec. 26, 2024

(54) METHOD AND SYSTEM FOR SELECTING A MASK

(71) Applicant: Fisher & Paykel Healthcare Limited, Auckland (NZ)

(72) Inventors: Benjamin Wilson CASSE, Auckland (NZ); Christopher Harding CAMPBELL, Auckland (NZ); Patrick Liam MURROW, Auckland (NZ); Matthew James MCCONWAY, Auckland (NZ); Clifton James HAWKINS, Auckland (NZ); Fahad Shams Tahani Bin HAQUE, Auckland (NZ)

(21) Appl. No.: 18/698,960

(22) PCT Filed: Oct. 6, 2022
(86) PCT No.: PCT/NZ2022/050127

§ 371 Date: Apr. 5, 2024

Related U.S. Application Data
(60) Provisional application No. 63/262,178, filed on Oct. 6, 2021.

(51) Int. Cl. G16H 20/40; G06V 40/16; G16H 10/20

(52) U.S. Cl. CPC G16H 20/40; G06V 40/165; G06V 40/166; G06V 40/171; G16H 10/20; G06V 2201/03

(57) ABSTRACT

A system for selecting a mask for a patient for use with a respiratory therapy device, the mask suitable to deliver respiratory therapy to the patient. The system comprises a processor configured to: receive data representing at least one digital image of a face of a patient; identify a predefined reference facial feature appearing in the image, the predefined reference facial feature being an eye of the patient; determine a measurement for the eye of the patient within the image; allocate a predefined dimension to the measurement, and determine a scaling factor for the image, the scaling factor being a ratio between the measurement and the predefined dimension; identify a further facial feature in the image; determine a measurement of the further facial feature in the image; and calculate a dimension of the further facial feature using the scaling factor and the measurement of the further facial feature; and, a memory for storing mask sizing data associated with patient masks; the processor further configured to: compare the calculated dimension of the further facial feature with the stored mask sizing data associated with patient masks and select a mask for the patient in dependence on the comparison.

### FIELD OF INVENTION

The present disclosure relates to a method and system for selecting a mask for a patient for use with a respiratory therapy device.

### BACKGROUND

The administration of continuous positive airway pressure (CPAP) therapy is common to treat obstructive sleep apnea. CPAP therapy is administered to a patient using a CPAP respiratory system which delivers therapy to the patient through a face mask. Different mask types are available to patients including full face masks, nasal face masks and under nose masks. The masks are typically available in different sizes to fit faces of different shapes and sizes. Correct fitting of masks is important to avoid leaks in the CPAP system which can reduce the effectiveness of the therapy. Poorly fitted masks can also be uncomfortable to the patient and result in a negative or painful therapy experience. Similar considerations are also taken into account when providing other pressure therapies via a mask e.g. BiLevel pressure therapy.

Masks are often fitted by medical professionals during the prescription of therapy. Often, patients have to go to an equipment provider or physician or sleep lab. The fitting process may be a trial and error process and can take an extended time period. More recently masks can be selected remotely by patients, for example via online ordering stores rather than physically purchasing the masks in an environment where the masks may be professionally fitted.

### SUMMARY OF THE INVENTION

In a first aspect the disclosure provides a method for selecting a mask for a patient for use with a respiratory therapy device, the mask suitable to deliver respiratory therapy to the patient, comprising the steps of:
receiving data representing at least one digital image of a face of a patient;
identifying a predefined reference facial feature appearing in the image, the predefined reference facial feature being an eye of the patient;
determining a measurement for the eye of the patient within the image;
allocating a predefined dimension to the measurement, and
determining a scaling factor for the image, the scaling factor being a ratio between the measurement and the predefined dimension;
identifying a further facial feature in the image;
determining a measurement of the further facial feature in the image; and calculating a dimension of the further facial feature using the scaling factor and the measurement of the further facial feature; and,
comparing the calculated dimension of the further facial feature with mask sizing data associated with patient masks; and,
selecting a mask for the patient in dependence on the comparison.

The measurement for the eye of the patient may be a width measurement. The measurement for the eye of the patient may be a height measurement.

The step of selecting a mask may comprise the step of identifying a mask.

The step of identifying an eye of the patient in the image may be performed by identifying at least two predefined facial landmarks in the image associated with the eye. The at least two predefined facial landmarks in the image may be the corners of the eye. The predefined facial landmarks may be the medial canthus and the lateral canthus. The measurement for the eye may be the width of the palpebral fissure.

The further facial feature may be identified by identifying at least two facial landmarks associated with the further facial feature. The further facial feature may be used to size the mask.

The step of determining a measurement of a facial feature may be performed by calculating a number of pixels of the image between at least two facial landmarks in the image associated with the facial feature.

The step of determining a measurement for the reference feature within the image may be performed by identifying two eyes of the patient within the image and calculating a measurement for each eye and calculating an average measurement for the two eyes.

The facial landmarks may be anthropometric features of a patient's face identified within the image.

The method may comprise the further steps of:
determining at least one attribute of the digital image;
comparing the at least one attribute with predefined attribute criteria; and determining whether the at least one attribute meets the predefined attribute criteria;
wherein the step of selecting a mask for the patient is performed in dependence on the at least one attribute meeting the predefined attribute criteria. The at least one attribute may comprise at least one of:
an angle of the face of the user within the image, the angle being at least one of the pitch angle, the yaw angle or the roll angle;
the focal length of the image;
depth of the patient's face in the image; and
at least one predefined landmark being identified in the image.

The at least one attribute may be the pitch angle, the predefined angle being between 0 to +−6 degrees with respect to the plane of the image.

The method may comprise the further step of providing feedback relating to whether the at least one attribute meets the predefined attribute criteria.

In embodiments, the step of calculating the dimension of the further facial feature may be performed for multiple images, to produce multiple calculated dimensions, the method comprising the further step of calculating an average dimension of the further facial feature across the multiple images; and using the average dimension to compare with the mask sizing data. The average dimension may be calculated across a predetermined number of images.

Embodiments may include the step of determining at least one attribute of the digital images;
comparing the at least one attribute with predefined attribute criteria; and determining whether the at least one attribute meets the predefined attribute criteria;
wherein the average dimension is calculated for images which meet the predefined attribute criteria.

Embodiments may comprise the further steps of:
presenting at least one user question to a user;
receiving at least one user response to the at least one user question; and
determining a mask category for the patient in dependence on the received user response.

The further facial feature may be selected from a plurality of facial features in dependence on the mask category.

The mask sizing data associated with patient masks may be associated with masks of the determined mask category.

Mask may be defined as being in a mask category, wherein different mask categories have different relationships between mask sizing data and dimensions of facial features.

The further facial feature may be selected from a plurality of facial features, the selection being made based on a designated mask category.

In a further aspect the disclosure provides a method for selecting a mask for a patient for use with a respiratory therapy device, the mask suitable to deliver respiratory therapy to the patient, comprising the steps of: presenting at least one user question to a user; receiving at least one user response to the at least one user question; determining a mask category associated with the user in dependence on the received user response; receiving a digital image of a face of a patient; within the image, identifying a predefined reference feature of the patient's face appearing in the image, allocating a dimension to the reference feature in the image, and determining a scaling factor for the image based on the reference feature; within the image, identifying at least one preselected feature of the patient's face appearing in the image, wherein the at least one preselected feature is selected in dependence on the determined mask type category, and calculating a dimension associated with the at least one preselected feature using the measurement scale; and, comparing the calculated dimension of the preselected feature with mask sizing data associated with patient masks and, selecting a mask for the patient in dependence on the comparison.

The calculated dimension of the preselected feature may be compared with mask sizing data associated with patient masks of the determined mask type category. Embodiments may determine if the preselected feature appears in the image and provide user feedback in dependence on whether it appears in the image.

In a further aspect the disclosure provides a method for selecting a mask for a patient for use with a respiratory therapy device, the mask suitable to deliver respiratory therapy to the patient, comprising the steps of:
receiving a digital image of a face of a patient;
determining attributes of the digital image;
comparing the attributes with predefined attribute criteria; and, provide user feedback relating to whether the attributes meet the predefined attribute criteria;
within the image, identifying a predefined reference feature of the patient's face appearing in the image, allocating a dimension to the reference feature in the image, and determining a measurement scale for the image using the reference feature;
within the image, identifying at least one preselected feature of the patient's face appearing in the image, and calculating a dimension associated with the at least one preselected feature using the measurement scale; and,
comparing the calculated dimension of the preselected feature with mask sizing data associated with patient masks; and,
selecting a mask for the patient in dependence on the comparison.

In a further aspect the disclosure provides a system for selecting a mask for a patient for use with a respiratory therapy device, the mask suitable to deliver respiratory therapy to the patient, the system comprising:
a processor configured to:
receive data representing at least one digital image of a face of a patient;
identify a predefined reference facial feature appearing in the image, the predefined reference facial feature being an eye of the patient;
determine a measurement for the eye of the patient within the image;
allocate a predefined dimension to the measurement, and
determine a scaling factor for the image, the scaling factor being a ratio between the measurement and the predefined dimension;
identify a further facial feature in the image;
determine a measurement of the further facial feature in the image; and calculate a dimension of the further facial feature using the scaling factor and the measurement of the further facial feature; and,
a memory for storing mask sizing data associated with patient masks;
the processor further configured to:
compare the calculated dimension of the further facial feature with the stored mask sizing data associated with patient masks and select a mask for the patient in dependence on the comparison.

The system may comprise a display to display the selected mask to the patient. The system may comprise an image capture device for capturing digital image data representing a face of a patient.

In a further aspect the disclosure provides a software application configured to be executed on a client device, the software application configured to perform the method of any of the previous aspects.

In a further aspects the disclosure provides a mobile communication device configured to select a mask for a patient for use with a respiratory therapy device, the mask suitable to deliver respiratory therapy to the patient, the mobile communication device comprising: an image capture device for capturing digital image data;
a processor configured to:
receive, from the image capture device, data representing at least one digital image of a face of a patient;
identify a predefined reference facial feature appearing in the image, the predefined reference facial feature being an eye of the patient;
determine a measurement for the eye of the patient within the image;
allocate a predefined dimension to the measurement, and
determine a scaling factor for the image, the scaling factor being a ratio between the measurement and the predefined dimension;
identify a further facial feature in the image;
determine a measurement of the further facial feature in the image; and calculate a dimension of the further facial feature using the scaling factor and the measurement of the further facial feature; and,
a memory for storing mask sizing data associated with patient masks;
the processor further configured to:
compare the calculated dimension of the further facial feature with the stored mask sizing data associated with patient masks and select at least one mask for the patient in dependence on the comparison; and
a user interface to display data related to the at least one selected mask.

In further aspects the disclosure provides a method for selecting a patient interface for a patient for use with a respiratory therapy device, the patient interface suitable to deliver respiratory therapy to the patient, comprising the steps of:
receiving data representing at least one digital image of a face of a patient;
identifying a predefined reference facial feature appearing in the image, the predefined reference facial feature being an eye of the patient;
determining a measurement for the eye of the patient within the image;
allocating a predefined dimension to the measurement, and
determining a scaling factor for the image, the scaling factor being a ratio between the measurement and the predefined dimension;
identifying a further facial feature in the image;
calculating a dimension for the further facial feature using the scaling factor; and, using the dimension to select a patient interface for the patient.

In further aspects the disclosure provides a system for selecting a mask for a patient for use with a respiratory therapy device, the mask suitable to deliver respiratory therapy to the patient, the system comprising:
a processor configured to
receive data representing at least one digital image of a face of a patient;
identify a predefined reference facial feature appearing in the image, the predefined reference facial feature being an eye of the patient;
determine a measurement for the eye of the patient within the image;
allocate a predefined dimension to the measurement, and
determine a scaling factor for the image, the scaling factor being a ratio between the measurement and the predefined dimension;
identify a further facial feature in the image;
calculate a dimension for the further facial feature using the scaling factor; and,
use the dimension to select a patient interface for the patient.

### BRIEF DESCRIPTION OF THE FIGURES

The ensuing description is given by way of non-limitative example only and is with reference to the accompanying drawings, wherein:

FIG. 1 is a schematic diagram of a respiratory therapy device including a blower for generating a flow of breathable gas, a conduit, and a patient interface for delivering the flow of breathable gas to the patient.

FIGS. 2A(i) and 2A(ii) are illustrations of a full face mask showing the mask positioned on the face and the contact area of the mask on the face.

FIGS. 2B(i) and 2b(ii) are illustrations of a nasal mask showing the mask positioned on the face and the contact area of the mask on the face.

FIGS. 3(i) and 3(ii) are illustrations of an under nose nasal mask showing the mask positioned on the face and the contact points of the mask on the face.

FIG. 4 is a schematic illustration of a mobile communications device.

FIG. 5 represents a basic architecture showing an interaction of a server with a mobile communications device.

FIG. 6 is a diagram showing facial features relating to an eye.

FIG. 7 is a flow chart showing steps performed in an embodiment.

FIG. 8 shows the alignment of a camera with the face of a patient when capturing an image for the mask sizing application.

FIG. 9 is an illustration of an image of a patient's face being displayed on the screen of the mobile communications device during image capture.

FIG. 10 is an illustration of an image of a patient's face identifying anthropometric features of the face.

FIGS. 11A and 11B are illustrations of an image of a patient's face identifying the eye width.

FIGS. 12A and 12B are illustrations of an image of a patient's face identifying various facial landmarks.

FIG. 13 is an example display of a mask recommendation to a patient.

FIG. 14 shows axes of rotation of the head, including pitch, yaw and roll.

FIG. 15 is a flow diagram showing the steps taken to analyse an image to determine if it meets various predefined criteria.

FIGS. 16, 16A, and 16B show image capture of a face of a patient and visual feedback provided to the patient.

FIGS. 17, 17A, and 17B show image capture of a face of a patient and visual feedback provided to the patient.

FIGS. 18, 18A, and 18B show image capture of a face of a patient and visual feedback provided to the patient.

FIG. 19 is a flow diagram showing steps performed by an embodiment.

FIG. 20 is an illustration of an example question displayed on a mobile communications device.

FIG. 21 is an illustration of a recommended mask displayed to a patient.

FIG. 22 shows example mask data scores for various questionnaire questions.

FIG. 23 shows the scores of a patient after completing a questionnaire.

FIG. 24 shows example relevant feature dimensions associated with fitting a full face mask.

FIG. 25 shows example relevant feature dimensions associated with fitting a nasal mask.

FIG. 26 shows example relevant feature dimensions associated with fitting an under nose nasal mask.

### DETAILED DESCRIPTION

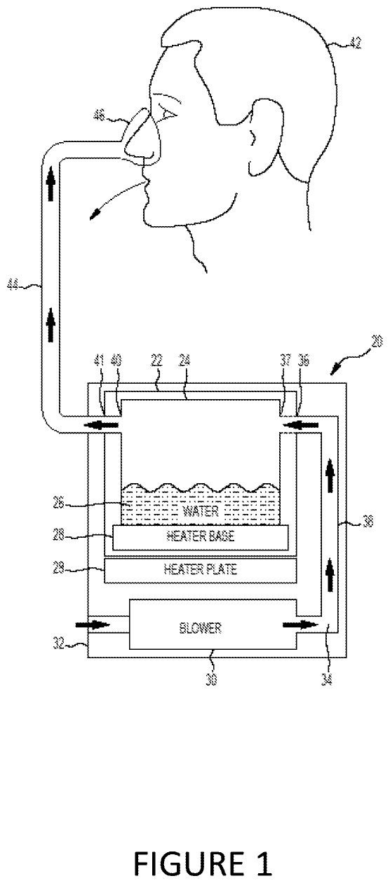

A method and system for selecting a mask for a patient for use with a respiratory therapy device are now described with reference to the accompanying FIGS. 1 to 26. The system for selecting the mask is configured to select a mask for a patient to use with a respiratory therapy device. The mask is automatically selected by capturing an image of a patient's face and determining dimensions of various features of the patient's face using a reference scale. Facial features may be defined between facial landmarks. The dimensions are compared with mask sizing data associated with different masks and mask sizes to automatically identify a suitable mask for the patient.

An exemplary embodiment will now be described in the following text which includes reference numerals that correspond to features illustrated in the accompanying figures.

FIG. 1 is a schematic illustration of a respiratory therapy device 20. The respiratory therapy device 20 can be used to provide CPAP (continuous positive airway pressure) therapy or BiLevel pressure therapy. The respiratory therapy device 20 including a humidification compartment 22 and a removable humidification chamber 24 that is inserted into and received by the compartment 22.

The humidification chamber 24 is inserted in a vertical direction when the compartment 22 is in an upright state. The compartment 22 has a top opening, through which the chamber 24 is introduced into the compartment 22. The top opening may have a lid so the humidification chamber 24 within the humidification compartment 22 may be accessed for removal for cleaning or filling. But this is optional, and other arrangements can be envisaged. For example, in other embodiments it is possible that the chamber 24 is inserted horizontally into the humidification compartment 22. Additionally/alternatively the respiratory therapy device may comprise a receptacle that includes a heater plate. The chamber is slidable into and out of the receptacle so that a conductive base of the chamber is brought into contact with the heater plate.

The humidification chamber 24 is fillable with a volume of water 26 and the humidification chamber 24 has, or is coupled to, a heater base 28. The heater plate 29 is powered to generate heat which is transferred to the heater base 28 of the chamber 24 (via the heat transfer plate 29) to heat the water 26 in the humidification chamber 24 during use.

The respiratory therapy device 20 has a blower 30 which draws atmospheric air and/or other therapeutic gases through an inlet and generates a gas flow 34 at an outlet of the blower 30. FIG. 1 illustrates an arrangement in which the outlet of the blower 30 is fluidly connected directly to a chamber inlet 37 via connecting conduit 38 and a compartment outlet 36. The chamber inlet 37 and the compartment outlet 36 may have a sealed connection when the humidification chamber 24 is in the operating position.

The gas flow 34 passes through the humidification chamber 24, where the humidity of the gas flow 34 is increased and exits via gases outlet 40 of the humidification chamber. The gas flow is delivered via a conduit 44 and a mask, cannula or similar patient interface 46 to a patient.

In the arrangement shown in FIG. 1, a chamber outlet 40 is sealingly connected to, or sealingly engaged with, a compartment inlet 41 by a sealed connection. In this embodiment, a lid to the compartment may or may not be provided.

In the arrangement of FIG. 1, the gas flow 34 passes through the humidification chamber 24, where the humidity of the gas flow 34 is increased and exits via chamber outlet 40. The chamber outlet 40 is sealingly connected to, or sealingly engaged with, a compartment inlet 41. It will be appreciated that in alternative embodiments, the chamber outlet 40 and the compartment inlet 41 need not be sealingly connected by a connector or otherwise sealingly engaged. The gas flow is delivered via a conduit 44 to a patient interface 46. The patient interface may be a mask. The patient interface may comprise one of: a nasal mask, an oral-nasal mask, an oral mask, a full face mask, an under nose mask, or any other suitable patient interface.

One or more sensors (not shown in FIG. 1) may be positioned within respiratory therapy device 20. Sensors are used to monitor various internal parameters of the respiratory therapy device 20.

Sensors (not shown) are connected to a control system (not shown) comprising a control unit. The sensors communicate with the control system. The control unit is typically located on a PCB. In one form the control unit may be a processor or microprocessor. The control system is able to receive signals from the sensors and convert these signals into measurement data, such as pressure data and flow rate data. In some forms, the control unit may be configured to control and vary the operation of various components of the respiratory therapy device to help ensure that particular parameters (such as, for example, air pressure, humidity, power output, blower speed) fall within desired ranges or meet desired ranges, thresholds or values. Typically, the desired ranges, thresholds or values are predetermined and are programmed into the control unit of the control system. Additional sensors, for example O2 concentration sensors or humidity sensors may be included into the respiratory therapy device. Further sensors may also comprise a pulse oximeter to sense blood oxygen concentration of a patient. A pulse oximeter is preferably mounted on patient and could be connected to the controller by a wired or wireless connection.

Blower 30 may control air and/or other gases flow in the respiratory therapy device. The control system and the control unit may be configured to control the state of blower 30 through transmission of control signals to blower 30. Control signals control the speed and duration of operation of blower 30.

Control system is programmed with multiple operating states for the respiratory therapy device. The control software for each operating state is stored within a memory within the control system. Control system executes the control software by transmitting control signals to the blower 30 and various other components of the respiratory therapy device to control the operation of the respiratory therapy device to create the required operating state.

Operating states for the respiratory therapy device may include respiratory therapy states and non-respiratory therapy states. Examples of respiratory therapy states include: CPAP (continuous positive airway pressure) commonly used to treat obstructive sleep apnea in which a patient is provided with pressurized air flow typically pressurized to 4-20 cmH20; NIV (non-invasive ventilation), for example biLevel pressure therapy, used for treatment of obstructive respiration diseases such as chronic obstructive pulmonary disease (COPD—which includes emphysema, refractory asthma and chronic bronchitis); high-flow; and, bilevel. Examples of non-respiratory therapy states include: an off state, in which the blower is off and provides no airflow through the respiratory therapy device; idle state, in which the blower is on and providing airflow through the respiratory therapy device but not providing therapy; and drying mode in which the blower may be on and cycle through a predefined speed pattern but not provide therapy. In drying mode a heater wire in the tube may be activated to a predetermined level e.g. 100% power and the blower may be activated to a preset flow rate or motor speed and driven for a predetermined time e.g. 30-90 mins. Drying mode dries out the conduit of any liquid or liquid condensate.

Different airflow conditions in the respiratory therapy device are required for different operating states. The control system provides control signals to the blower 30 to control blower operating parameters, including activation and speed, to provide the required airflow conditions in the respiratory therapy device.

Software programs defining the operating conditions required for the various operating states of the respiratory therapy device are stored within memory of control system. During operation of a particular operating condition, the control system receives signals from various sensors and components of the respiratory therapy device at a communication module 62 defining the conditions within the respiratory therapy device, for example pressure data and flow rate data. The control system 60, and in particular processor, is configured to compare the conditions within the respiratory therapy device with predefined operating conditions for the operating state and to control and vary the operation of various components of the respiratory therapy device to help ensure that particular conditions (such as, for example, air pressure, humidity, power output, blower speed) fall within desired ranges or meet desired thresholds or values associated with the required operating state. The desired ranges, thresholds or values are predetermined and programmed into the software program.

In some embodiments, the respiratory therapy device includes a transceiver to transmit and receive radio signals or other communication signals. The transceiver may be a Bluetooth module or WiFi module or other wireless communications module. The transceiver may be a cellular communication module for communications over a cellular network e.g 4G, 5G. In one example the transceiver may be a modem that is integrated into the device. The transceiver allows the device to communicate with one or more remote computing devices (e.g. servers). The device is configured for two way communication (i.e. to receive and transmit data) to the one or more remote computing devices (e.g. servers). For example device usage data can be transmitted from the device to the remote computing devices. In another example therapy settings for the device may be received from the one or more remote computing devices. In a further example the respiratory therapy device may comprise multiple transceivers e.g. a Wifi module, a Bluetooth module, and a modem for cellular communications or other forms of communication.

In some embodiments the transceiver may communicate with a mobile communications device.

The patient interface 46 is typically a mask configured for connection to the patient's face. The mask may be held in place on the face of the patient using a headband which extends around the head of the patient. Other suitable means for holding the mask in place may also be used, for example adhesives or suction. The mask is an important part of the respiratory system and preferably provides comfortable delivery of gas to the patient without leakage. CPAP masks have bias flow holes to allow exhaled gases to escape the mask. Different mask types are available to patients including full face masks, nasal face masks and under nose nasal face masks. The masks are typically available in different sizes to fit faces of different shapes and sizes. Correct fitting of masks is important to avoid leaks in a CPAP system which can reduce the effectiveness of the therapy or respiratory support delivered via the mask. Poorly fitted masks can also be uncomfortable to the patient and result in a negative or painful therapy experience, for example by causing pressure sores on sensitive parts of the face. Selecting the correct mask for a patient is critical to providing reliable and ongoing therapy.

A number of factors are relevant when selecting a mask for a patient:

A first consideration is selecting the correct mask category for a patient. Patients breathe in different ways, some patients breathe through their nose, some patients breathe through their mouth, and, some patients breathe in a combination through their nose and mouth. Optimal respiratory therapy or respiratory support can be provided to a patient by prescribing a mask type suitable to the way a patient breathes. The main mask categories are: full face mask, nasal mask, under nose nasal mask. Other types of masks include oral masks (seal around the mouth only), hybrid masks (seals around the mouth and has nasal pillows to seal with nostrils), full face mask variation (seals around mouth and under nose but not pillows), masks that seal at least partly with the mouth and/or at least partly with the nares. Each mask functions to create a seal with either the mouth, nose, or both to maintain effective delivery of pressure-based therapy e.g. CPAP. The consideration of which mask a patient should use is influenced by which airway(s) they predominantly breathe from—that airway is where pressure-based therapy should be delivered to keep the tissue of the main airway open and prevent collapse. The chosen mask seals against the airway and essentially extends the airway fluidically to the therapy device which supports breathing E.g. if the patient predominantly breathes from their nose then they will receive the most effectively respiratory aid if a nasal mask, under nose mask or nasal pillows are used to seal with that airway and provide pressure.

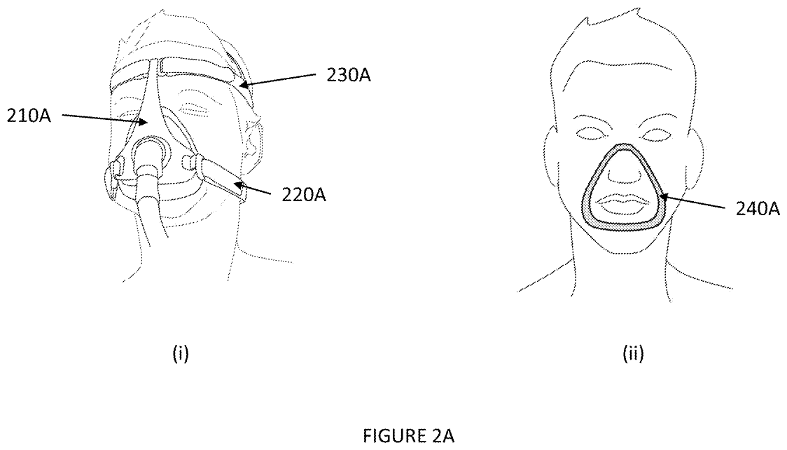

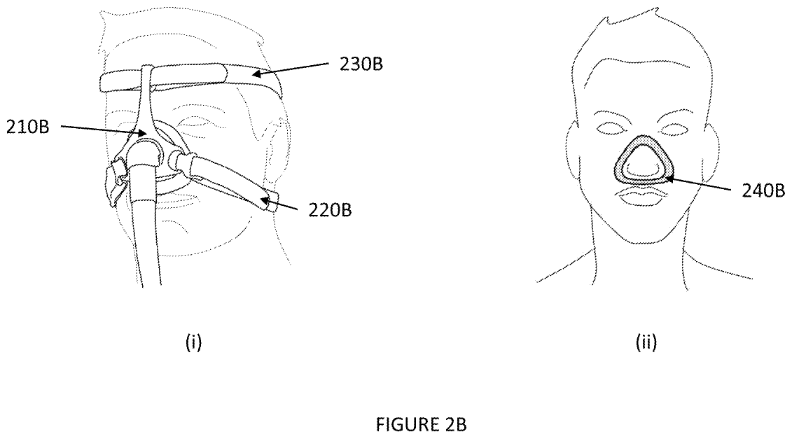

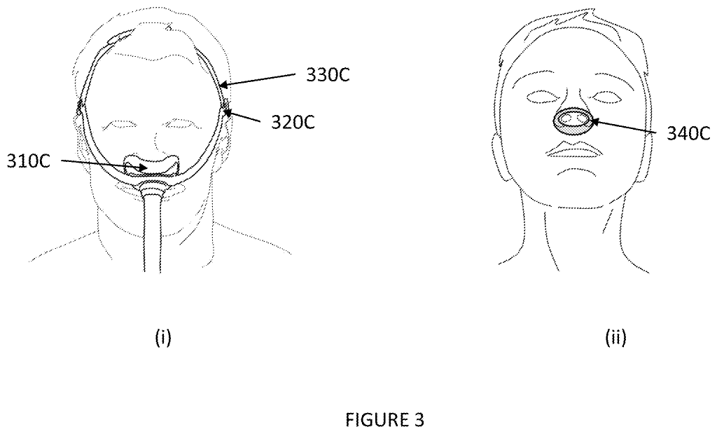

Examples of different mask categories are shown in FIGS. 2 and 3. FIG. 2 illustrates each mask category on the face of a patient and, separately, illustrates the contact area for each mask category on the face of the patient.

FIG. 2A shows a full face mask 210A which covers the nose and mouth of the patient. Full face mask 210A is held to the face of the patient using headgear. Headgear includes a strap 220A extending around the jaw and/or cheek and neck of the patient and a second strap 230A extending around the top of the head of the patient. Full face masks seal around the whole mouth and nose region and over the nose bridge. As illustrated in FIG. 2A(ii), seal 240A extends under the mouth of the patient, around the sides of the nose and over the nose bridge. The flexible seal of a full face mask can conform/mould to varying surfaces around the nose and mouth to create an effective seal to maintain pressure when therapy is delivered.

FIG. 2B shows a nasal face mask. Nasal face masks are the same as nasal masks. The terms can be interchangeably used. The nasal face mask covers the nose only and does not cover the mouth. Nasal face mask 210B is held to the face of the patient using a strap 220B extending around the jaw and/or cheek and neck of the patient and a second strap 230B extending around the top of the head of the patient. Nasal face masks seal around the nose region and over the nose bridge. As illustrated in FIG. 2B(ii), seal 240B extends around the nose of the patient. It seals under the nose of the patient, under the nostrils and above the mouth, around the sides of the nose and over the nose bridge. The flexible seal of a nasal face mask can conform/mould to varying surfaces around the nose to create an effective seal to maintain pressure when therapy is delivered.

FIG. 3 shows an under nose nasal mask. Under nose nasal masks only seal with the nostrils. This is a less intrusive way to create a nasal seal than using a nasal mask. The under nose nasal mask 310C is held to the face of the patient using a strap 320C extending around the back of the head of the patient and a second strap 330C extending over the top of the head of the patient. Under nose nasal masks seal around the nose region only. As illustrated in FIG. 3(ii), seal 340C extends around the nostrils of the patient. The flexible seal of an under nose nasal mask can conform/mould to varying surfaces around the nose to create an effective seal to maintain pressure when therapy is delivered. The seal is created on a portion of the underside of the nose of the patient. The seal 340C may also seal up around the sides of the nose or may seal around the side of the nose e.g. within a region of the alar crease or about the alar of the patient. The flexible seal of an under nose nasal mask can conform/mould to varying surfaces around the nose to create an effective seal to maintain pressure when therapy is delivered.

Under nose full face masks cover the mouth and seals under nose. Sizing for under nose full face masks use the sizing guide for the under nose nasal mask using the under nose nasal mask sizing parameters in combination with mouth width.

Within each mask category, masks may be provided in different sizes, for example XS, S, M, L. The size of the mask is generally defined by the seal size, i.e. the size of the mask seal that contacts the face. Generally, patients with larger heads require a larger seal size in order to provide an optimal or working seal. The size of the headgear is also a consideration for effectiveness and comfort and the headgear may also be provided in different sizes depending on the size of the head of the patient. Some mask categories may also include an XL mask size.

When selecting a mask for a patient, further considerations can be taken into account relating to the sleeping habits of the patient. Typical prescriptions for respiratory therapy require the patient to wear the mask throughout the night while sleeping. Factors including patient movement during the therapy session, for example whether the patient is a restless sleeper, and also whether the patient wears glasses in bed, are also factors to be considered when selecting a mask for a patient, in order to optimize the effects of the therapy and a patient's ongoing adherence to a therapy program. Other considerations include, safety: poorly fit masks may lead to a patient tampering with the fit and settings etc. Leaks may also be noisy and disrupt sleep (of the patient and partner). Other considerations may also be taken into account as some OSA patients may have other health issues.

When selecting a mask for a patient, objectives include minimize leakage between the mask and the face in order to optimize therapy but also to avoiding patient discomfort by avoiding excessive pressure around the contact area of the mask with the face. Poorly fitting masks or masks which do not match the patient's breathing type can affect the effectiveness of therapy, patient comfort and patient therapy adherence.

Typically, masks are fitted by clinicians during patient diagnosis. Mask fitting is typically performed in person with the patient able to try on different mask types and sizes in order to select the most appropriate mask type and mask size for the patient under the guidance of a professional. Clinicians are technical experts and experienced with mask fitting for patients.

Masks are consumable products with a limited lifetime of optimal usage and typically a patient needs to replace a mask every few months. There has been a desire for remote ordering of masks by patients. Additionally, some patients prefer to select a mask without visiting a clinician.

Recently, mask suppliers have begun to offer remote mask selection and remote mask ordering options to patients. These options may allow a patient to view a catalogue of masks, select a mask from the catalogue and order the mask remotely, for example over the internet. One challenge with allowing patients to select a mask is that the fitting procedure is not undertaken by technical experts and so the mask selected by the patient may not be optimal in terms of mask category or mask fit. As discussed above, poorly fitting masks or mask which do not match the patient's breathing style and/or other sleeping factors, for example the position in which a patient tends to sleep e.g. side sleeper, can result in sub-optimal therapy and discomfort to the patient. These factors may reduce therapy results and can result in poor patient therapy adherence.

Automatic mask sizing software applications which collect patient data and recommend masks to patients have been developed. These can provide improved results compared to independent patient selection of masks. However, one of the challenges of automatic mask selection is the capture of accurate patient facial measurement data to allow the software application to identify a mask which fits the patient. These software application often require significant processing. Software applications for recommending masks to patients often provide unreliable measurement data or rely on patient expertise or input to retrieve measurements. These factors can result in the recommendation of sub-optimal masks to the patient.

Another challenge is to make the process simple to use and fast in addition to providing accurate measurements and sizing. Patients may be unfamiliar with technology or have limited mobility, and hence there is a need for a simple, intuitive sizing process.

In an embodiment, a method and system for selecting a mask for a patient for use with a respiratory therapy device or system is provided. The mask is suitable to deliver respiratory therapy or respiratory support to the patient. The method comprises the steps of receiving data representing at least one digital image of a face of a patient. The method identifies a predefined reference facial feature appearing in the image, where the predefined reference facial feature is an eye of the patient. The method determines a measurement for the eye of the patient within the image and allocates a predefined dimension to the measurement. The method determines a scaling factor for the image, where the scaling factor is a ratio between the measurement and the predefined dimension. The method identifies a further facial feature in the image, determines a measurement of the further facial feature in the image and calculates a dimension of the further facial feature using the scaling factor and the measurement of the further facial feature. The method compares the calculated dimension of the further facial feature with mask sizing data associated with patient masks and selects a mask for the patient in dependence on the comparison.

Embodiments provide an accurate measurement system that allows a non-technical expert, to accurately and reliably capture the information required for the system to recommend a well-fitting mask. The method can be implemented, using non-professional equipment. Embodiments capture images of the patient face which allow accurate and reliable sizing to be derived using a reference scale. The described method and system provide a convenient method for mask sizing as a user (e.g. an OSA patient) can perform this method at home without having to visit a clinician and without the need of any professional equipment. Further the method for sizing is convenient as it can be executed on a mobile device of a user e.g. a smartphone or tablet. The described method and system for mask sizing are also advantageous because there is no requirement for a separate reference object that needs to be held in front of the patient's face to perform the mask sizing.

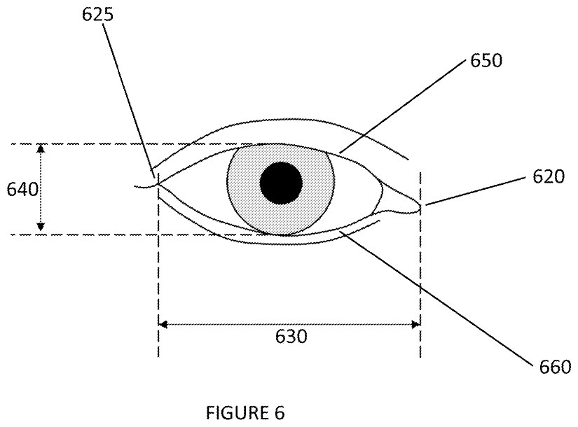

In the following described exemplary embodiment, the reference facial feature used to scale an image of the patient's face is the eye. FIG. 6 shows a human eye and surrounding parts of the face. The eye includes two corners: a first corner 620 positioned on the face at an innermost point of the eye, closest to the centre of the face; and a second corner 625 positioned on the face at an outermost point of the eye, furthest from the centre of the face. The distance between the corners of the eye is the eye width.

These corners may be defined by the two canthi of the eye. The facial landmark relating to the innermost point of the eye is the medial canthus 620. The facial landmark relating to the outermost point of the eye is the lateral canthus 625.

The width of the eye is a useful feature to use as a reference feature of the face because its dimension is found to have minimal variance amongst adults, typically aged 16 and above.

In one example, the width of the eye is the distance between the corners of the eye.

In other embodiments, the width of the eye is the distance between the medial canthus 620 and the lateral canthus 625.

In other examples, the width of the eye may be defined as the distance of the white region of the eye, where the corners 620 625 are defined as the point of contrast between the white of the eye and the face.

In other examples, the width of the eye is the horizontal distance between the medial canthus 620 and the lateral canthus 625. This distance is the horizontal palpebral fissure 630. The horizontal palpebral fissure is a useful feature of the face to use as a reference feature. This feature is found to have minimal variance amongst individuals aged 16 and above. The horizontal palpebral fissure is generally consistent between males and females and also is generally consistent for different ethnicities. In other examples the height of the eye may be used as a reference feature. The height of the eye may be defined as the distance between the upper eyelid 650 and the lower eyelid 660 when the eye is open. The height of the eye may be the maximum distance between the upper eyelid and the lower eyelid when the eye is open. This height may be defined as the vertical palpebral fissure 640.

The eye width can be detected in images or videos of a patient's face. Since the canthi are landmarks of the face, rather than parts of the eyeball, like the iris or the pupil, these landmarks are not obscured by the eye lid of the patient. Since the canthi are landmarks of the face, the eye width can be captured in an image even when the eye is closed, partly closed or during blinking. These landmarks can be detected more easily than the iris and parts of the eyeball. Detection of parts of the eyeball, like the iris or pupils, may also be difficult due to reflection from light sources or due to shadows cast from eyelids or eyebrows. Parts of the eyeball may also be obscured by the eyelid. The width of the eye is a greater length than other parts that may be used as reference features, for example the iris or the pupil, so any percentage measurement error will likely be lower than for a smaller reference feature. Similarly, the eye height can be detected in images or videos of a patient's face.

A further benefit of using the width of the eye, or height of the eye, as a reference feature is that measurements can be obtained for both eyes of a patient within an image, allowing an average measurement to be calculated. This averaging can also reduce the error in the measurement value.

Embodiments of the invention provide a method and system for selecting a mask for a patient for use with a respiratory therapy device. The mask is suitable to deliver respiratory therapy to the patient. The system receives facial images of the patient and uses the facial images to select a mask for the patient. The system extracts dimensions of relevant features of the patient's face from the images and selects an interface for the patient that will fit the various dimensions of the patient's face.

Facial images are digital images that include the face of the patient.

The methods may be implemented on an user device. A software application may be loaded onto a user device, for example a mobile phone, tablet, desktop or other computing device. The software may operate solely on the user device or may be connected to a server across a communications network.

In a first exemplary embodiment now described, the method is implemented by a software application executed on a mobile communications device. The terms mobile communication device, mobile communications device, user device and mobile device are used interchangeably.

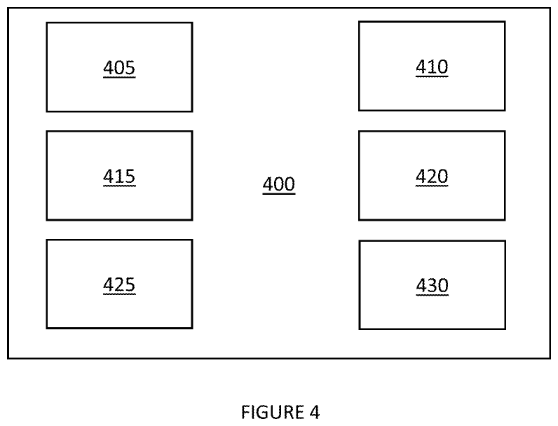

A schematic representation of the mobile communications device is shown in FIG. 4. Mobile communications device 400 includes an image capture device 405. In the example of FIG. 4 the image capture device is a digital camera. Mobile communications device 400 includes memory 420. Memory 420 is a local memory within communication device 400. Memory 420 is suitable for storing software applications for execution on the mobile communications device, algorithms and data. Data types include mask data including mask category data and mask sizing data, reference scales and dimension information for facial features and landmarks, image recognition software applications suitable for identifying facial features and landmarks within images, questions for presentation to the user, etc.

Mobile communications device 400 includes processor 410 for executing software applications stored in memory 420. The mobile communications device includes display 430. The display is suitable for presenting information to a user, for example in the form of text or images, and also for displaying images captured by camera 405. User input device 425 receives input from a user. User input device may be a touch screen or keypad suitable for receiving user input. In some embodiments user input device 425 may be combined with display 430 as a touch screen. Other examples of user input devices include microphones. Microphones receive voice commands or other verbal indicators from the patient.

Transceiver 415 provides communication connections across a communications network. Transceiver 415 may be a wireless transceiver. Transceiver 415 may support short range radio communications, for example Bluetooth and/or WiFi. Transceiver 415 also supports cellular communications. Alternatively multiple transceivers may be implemented, each transceiver configured to support a specific communication method (i.e. communication protocol), such as for example WiFi, Bluetooth, cellular communications etc.

In the following example mobile communications device 400 is a mobile phone but device 400 could be a tablet, laptop or other mobile communications device having the components and capabilities described with respect to FIG. 4. In some illustrated examples the mobile communications device is a smartphone.

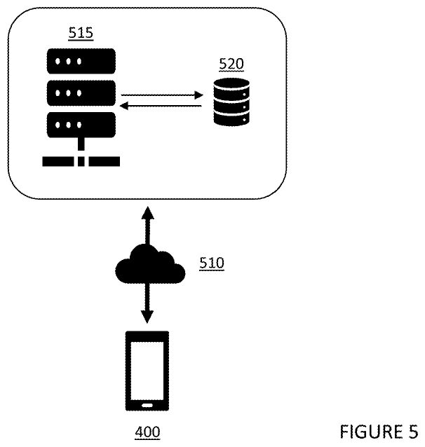

The communication path between mobile communications device 400 and various servers is shown in FIG. 5. In FIG. 5 mobile communications device 400 communicates with server 515 across a communications network 510. Server 515 accesses and/or communicates with database 520. The mobile communications device 400 exchanges data with sever 515 and database 520. Communications device 400 may request data from server 515 and/or database 520. Communications device 400 may provide data to server 515 and/or database 520. Server 515 and/or database 520 may provide data to mobile communications device 400 in response to a request from mobile communications device and/or may selectively push data to mobile communications device 400.

Network servers typically provide mobile communications device 400 with updates. The updates may relate to data updates for look up tables and other databases stored in memory 420. Updates may relate to the patient interface fitting application, providing changes to the software application to change or improve the operation of the application.

The method of patient interface fitting may be performed on the mobile device 420 or may be performed across a distributed computer system. When executed on the mobile communications device, all processing, image capture, data storage and recommendations is performed on the mobile communications device. The application can operate offline without a communications connection to external severs. When the method is performed using a distributed computing system, functionality performed during the method may be performed on different devices or at different locations. Data may be stored in different locations and retrieved or provided across communications networks. In some examples, application may be run entirely on a remote server using data stored in remote databases, in an in the cloud configuration

Data relating to the mask selection software application may include: questions to be presented to a patient during a mask selection process within a patient questionnaire; database data associating responses to questionnaire questions to various mask categories; data relating to sizing information associating facial feature dimensions with mask sizes; and, general information about devices or masks, for example mask instructions, cleaning instructions, FAQs and safety information. Details of some specific databases used in various embodiments are provided below. The diagram of FIG. 5 is for illustrative purposes only, further implementations may include communication connections between multiple servers and databases.

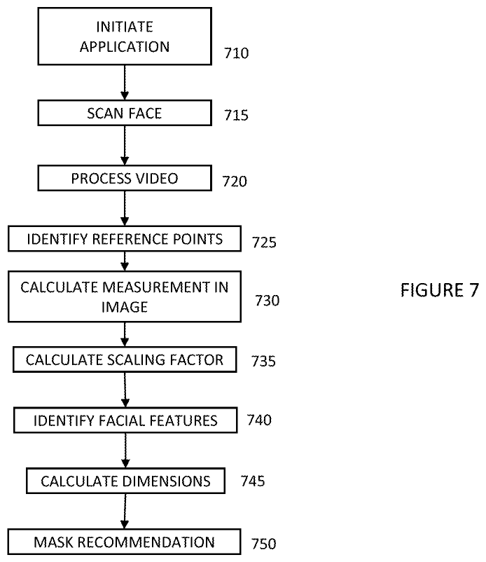

The steps performed by a mask selection software application operating on a mobile communications device are now described with reference to FIG. 7. In the description the terms: mask selection software application; mask sizing application; software application; and, application, are used interchangeably. The mask selection software application is a software programme that may be stored in memory 420 and executed by processor 410. The software programme is a computer executable programme for execution using the processor 410 of mobile communications device 400. The computer programme may include a series of instructions to be executed by processor 410 and may be or may include algorithms. The programme is executed locally using data that is acquired at the mobile communications device 400. In the following description, the various modules, for example facial detection module, face detection module and face mesh module, the applications, and the algorithms, may specifically form part of the mask selection software application or may reside as separate computer programmes stored in memory 420 which are called by the mask selection software application during execution when required.

At 710 a mask selection software application is opened on mobile communications device 400. The mask selection software application is opened for the purpose of recommending a respiratory therapy mask to a patient. The mask selection software application is a software programme that may be stored in memory 420 and executed by processor 410.

On selection of the mask selection software application by the patient, the mask selection software application is initiated at 710. The mask selection software application accesses camera 405 in order to capture a digital image of the patient's face by scanning at 715. Preferably the forward facing camera on the same side of the device as the display screen is accessed by the mask selection software application. This orientation is commonly recognized as capturing an image in ‘selfie’ mode, so the patient can view the image on the display screen during image capture. The mask selection software application may provide guidance to the patient, for example in the form of text instructions or example images on the display screen 430, to help the patient capture a suitable image. In other examples, the rear face camera is used for image capturing. This may facilitate use of the mask sizing app by a clinician sizing a patient. This allows the patient to have someone assist them in capturing a facial image.

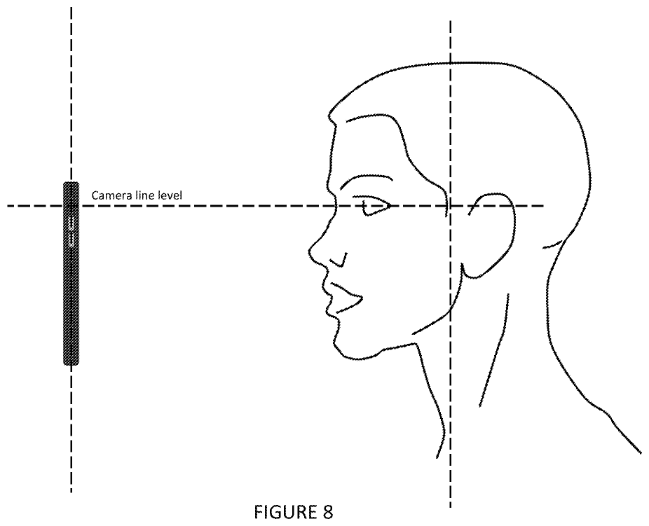

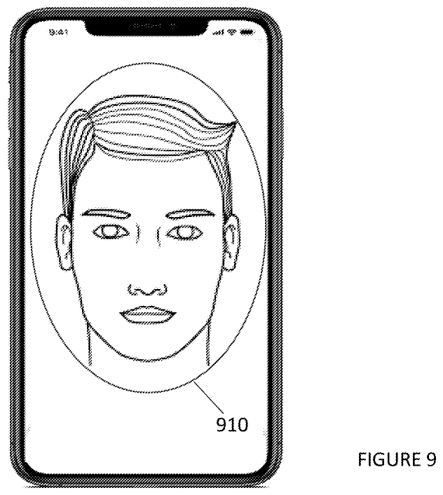

The mask selection software application is configured to be operated independently by a patient and so an image of the patient's face may be obtained by holding the mobile communications device away from the patient with the camera directed at the patient's face, as shown in FIG. 8. Preferably the image captured by the camera is displayed to the user on display screen 430 as shown in FIG. 9. Visual guidance to aid the patient in capturing the image may be provided, for example in the form of frame 910. Further guidance which may include text may be presented on the screen instructing the user to position their face within the frame.

During image capture, the application captures a stream of digital image frames. The rate at which frames are captured may vary between applications or devices. The rate at which frames are captured may be related to the clock in the mobile device and may be dependent on the type of mobile device. In some embodiments only a single image frame is captured. In such systems the application may prompt the patient to capture the image, for example by providing a button on the screen for taking the image. In other embodiments multiple frames are captured as part of a video in a frame sequence. Individual or multiple frames may be extracted from the multiple frames for analysis. In exemplary systems, multiple frames are automatically captured. The video image frames or image frame is captured at 720 and processed to produce a digital image file of the face of the patient. The file may be any suitable file type, for example JPEG. Alternatively there is no capturing per se i.e. the processing can be done on the image frame itself taken from the image buffer. In such an example there will be no specific image file like a jpeg created.

The mask selection software application includes a facial detection module. The facial detection module is a software programme configured to analyse an image file and detect predefined facial landmarks in the image. At 725 the mask selection software application runs a facial detection module on the image. The mask selection software identifies facial landmarks. In some implementations, no actual JPG is produced but rather the software uses a matrix or array of data for e.g. of pixel values and stores that in temporary memory. Preferably no permanent record of the images is stored or transmitted as the processing is done locally. The image may be cached and processed and then deleted. This is to respect privacy of the users and to provide trust to user's that their facial data is not being transmitted etc.

In exemplary embodiments the facial detection module is a machine learning module for face detection and facial landmark detection. The facial detection module is configured to identify and track landmarks of the face. Preferably the facial detection module operates in real time and analyses images generated by the camera of the mobile device as they are captured.

Exemplary facial detection modules may comprise a face detection module and a face mesh module. The face detection module allows for real time facial detection and tracking of the face. The face mesh module provides a machine learning approach to detect the facial features and landmarks of the user's face. The machine learning approach continually updates its libraries, and uses stored data on a plurality of sampled faces to correct for irregularities in a captured image. The face mesh module provides locations of face landmarks and provides a coordinate position of each landmark. The landmark positions are provided as a coordinate system. For example the coordinate system may be a cartesian coordinate system or a polar coordinate system. The zero point i.e. reference point for the coordinate system is preferably located on the patient's face e.g. at the center of the nose. Alternatively, the reference point may be located off the face i.e. a point in space that is used by the module when determining the locations of the facial landmarks and providing location information e.g. coordinates. The face detection module and the face mesh module together allow for tracking of landmarks and features. These may be two separate programmes or may be incorporated into a single programme or algorithm. Alternatively, the face detection module and face mesh module may be separate computer programs i.e. that may be stored in the memory of the mobile communication device. The processor 410 is configured to execute the programs in this alternative configuration.

Exemplary embodiments may be configured to select a predefined subset of the total facial landmarks detected by the facial detection module and to calculate dimensions for features defined by these landmarks only. The particular subset of the total facial landmarks may be selected based on a current operation of the mask selection software application, patient input, mask category or other selection criteria.

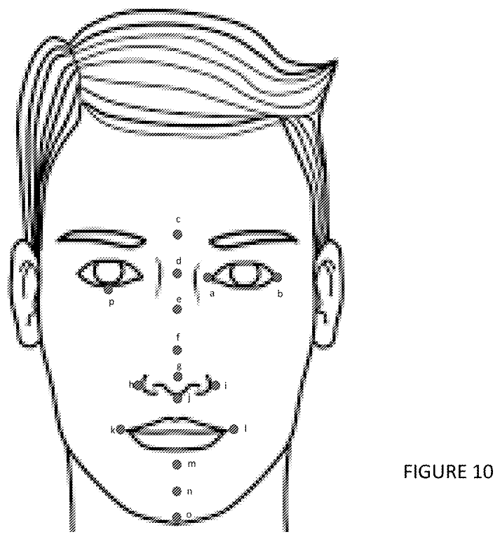

FIG. 10 is an illustration of a patient's face identifying various facial landmarks. Facial landmarks are points of the face. These facial landmarks are anthropometric landmarks of the face, including for example but not limited to:
a) Medial canthus
b) Lateral canthus (i.e. ectro canthus).
c) Glabella
d) Nasion
e) Rhinion
f) Supratip lobule
g) Pronasale
h) Left alare (alar lobule)
i) Right alare (alar lobule)
j) Subnasale
k) Left labial commissure (i.e. left corner of mouth)
I) Right labial commissure (i.e. right corner of mouth)
m) Sublabial
n) Pogonion
o) Menton
p) Orbitale

Facial features are defined by facial landmarks. For example, the facial features may be located between facial landmarks. The dimension of the facial feature may be defined as the distance between certain facial landmarks. For example, the facial feature of nose width is defined between the left and right alar lobule (landmarks h and i of FIG. 10). Nose width may be calculated as the distance on the face between the left and right alar lobule. Nose width may be calculated when the coordinates of the left and right alar lobule are known.

At 725 the application identifies predefined facial landmarks in the image captured by the patient device. The application applies a coordinate system onto the digital image of the patient's face. In an exemplary embodiment, the coordinate system is a 3-dimensional coordinate system (x, y, z). In one implementation the centre of the nose is set as coordinate (0,0,0) and the coordinates of all landmarks are determined in relation to the (0,0,0) point.

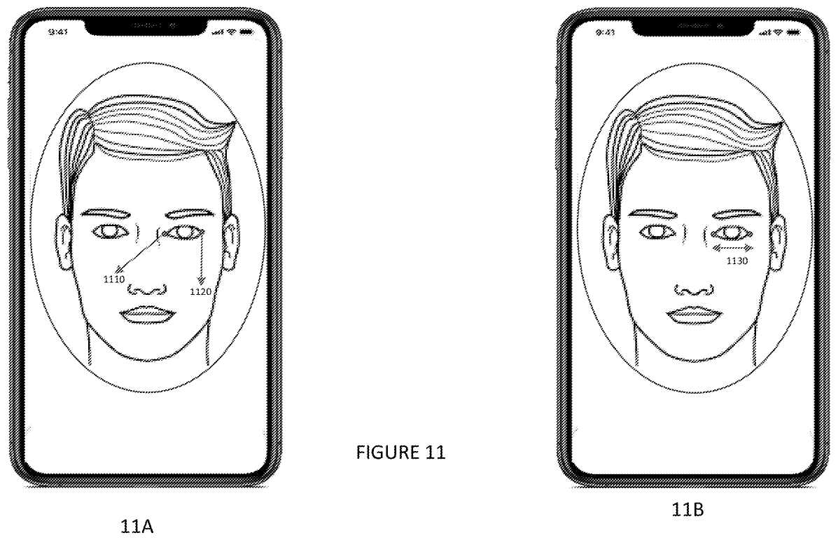

As shown in FIG. 11A, the application identifies the medial canthus 1110 and the lateral canthus 1120 within the image of the patient's face, i.e. the two corners of the eye of the patient. The x, y, z coordinates for the medial canthus and the lateral canthus are identified, lateral canthus (x1, y1, z1) and medial canthus (x2, y2, z2).

Now shown in FIG. 11B, a measurement for the reference feature of the eye width 1130 is calculated within the image. In this exemplary embodiment, the measurement for the eye width is calculated using the x and y coordinates only, z coordinates are ignored. In other embodiments, the z coordinates may also be used in calculating the measurements.

In the exemplary embodiment, the measurement for the eye width is calculated between the canthi using the formula:

$$Eye width measurement = \sqrt{{( x 1 - x 2 )}^{2} + {( y 1 - y 2 )}^{2}}$$

The measurement is the length of the feature in the image. The units of the measurement may be pixels of the image. Other units for the measurement, for example image vectors may be used. Calculations based on two dimensions (x and y coordinates) only can be useful as it saves on computation.

Further embodiments calculate the eye width measurement using the x coordinates of the canthi only. In these exemplary embodiments the eye width measurement is calculated using the formula, |x1-x2| or |x2-x1|. In some embodiments it may be useful to use more than one of the x, y, and z coordinates to account for any non-standard positioning of facial features.

The application may calculate the width of one eye in the image at step 730 as described above. In further embodiments, the application identifies the corners of both eyes of the patient's face appearing in the image. A width measurement is calculated for each eye and averaged in order to obtain an average eye width for the patient in the image. Use of an average width across both eyes can reduce errors.

At 735, a scaling factor for the image is calculated. Memory 420 stores a reference dimension associated with the eye. As discussed above the eye width is a useful reference feature as it shows minimal variance across adults. The dimension is the size of the feature on the patient's face. Exemplary embodiments use the reference dimension of the eye width to be 28 mm. The reference dimension may relate to the average eye width (i.e. horizontal palpebral fissure) of a human eye. A different reference dimension may be used for the height of the eye, for example 10 mm. This corresponds to the average eye height (i.e. vertical palprebal fissure). In the illustrated and described sizing method eye width is used. Other embodiments may select alternative reference dimensions for the eye width, for example 29 mm.

The application calculates a scaling factor for the image using the eye width measurement in the image and the eye width dimension of 28 mm. The scaling factor is the ratio between the width measurement in the image and the width dimension. As discussed above, the width measurement may be taken in pixels or in some other suitable units.

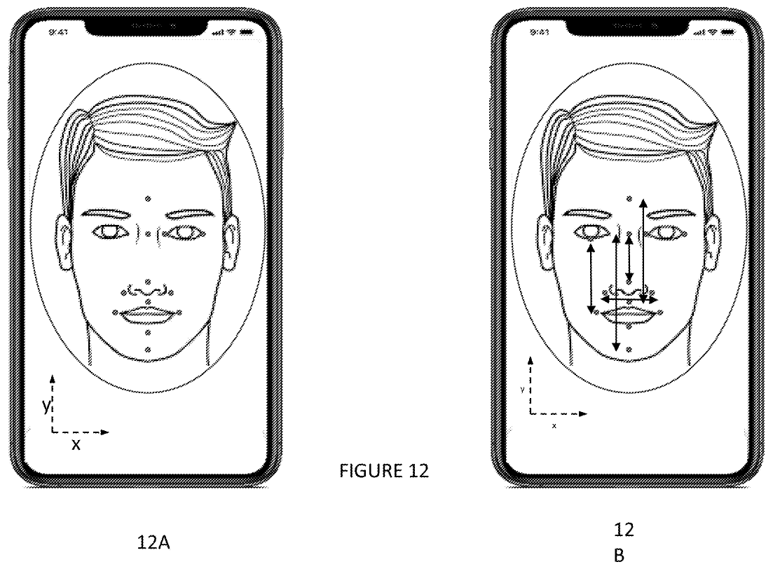

Referring to FIG. 12A, at 740 facial landmarks are identified in the image by the facial detection module and the coordinates of each facial landmark (x, y, z) in the image are determined. The processor of the mobile device is configured to receive image coordinates for each of the identified facial landmarks. The anthropometric landmarks of interest may be a preselected subset of the total anthropometric landmarks identified in the image.

Referring to FIG. 12B, the measurements of preselected facial features are calculated by identifying the two anthropometric landmarks associated with each preselected facial feature and determining the length between the landmarks in the image. This measurement may be the difference between the absolute value of x coordinates (e.g x1-x2) only or the absolute value of y coordinates (y1-y2) only. The horizontal dimension i.e. x dimension may be obtained by determining the difference between the x coordinates and the vertical dimension i.e. y dimension may be obtained by determining the difference between the y coordinates (as described earlier). Alternatively, the measurement between the landmarks may be calculated using the equation \sqrt{(x1-x2)²+(y1-y2)²}. Exemplary embodiments may calculate the measurements using two dimensions or three dimensions. Again, the measurements may be calculated in pixels or any other suitable unit of measurement.

In FIG. 12B, the arrows illustrate measurements of various facial features that may be calculated. The z dimension may be used for example to calculate nasal depth e.g. the z distance between the subnasale and pronasale. The measurements of the nasal features are calculated in pixels or some other measure (e.g. image vectors). The z dimension may only be relevant for particular mask categories, for example the under nose mask shown in FIG. 3. The z depth measurement |z2-z1| or |z1-z2| is calculated in the image and may be converted to a facial dimension for the patient using the same scaling factor derived from the eye width as previously described.

At 745, the facial measurements in the image, i.e. the number of pixels, is converted to a facial dimension using the scaling factor for the image calculated with respect to the eye width dimension. For example, using 28 mm as the dimension of the eye width:

$$facial feature dimension = \frac{facial feature measurement ( pixels ) \times 28 mm}{reference feature measurement ( eye width measure in pixels )}$$

Optionally each of the measurements may be multiplied by a scaling factor. The scaling factor is a suitable scalar that is predetermined. In some embodiments the scaling factor may compensate for a fish eye effect of camera lenses and/or other distorting factors.

The feature identification and dimension calculations may be calculated from a single image. In another embodiment, multiple images may be captured by the camera, each image being a separate image frame, and processed. In each image, the dimensions may be calculated for each feature and the final calculated dimension for a feature on the face of the patient is an average dimension across the multiple images, to reduce errors.

The facial detection module may be preprogramed to capture a minimum number of frames to calculate an average dimension across. In an exemplary embodiment at least 30 frames are captured and/or processed. In another example, at least 100 frames are captured and/or processed. The facial detection module may be preprogramed to require data to be captured over a minimum length of time, for example 10 seconds of video, to be captured and processed i.e. 10 seconds of x, y, z data of facial landmarks. Measurements are then averaged over the captured frames.

In order to manage memory storage space, frames or patient images may not be stored in the memory, i.e. nothing persists. The frames are stored for the time to process and then deleted. Temporary memory could be ROM, RAM and optionally some temporary cache memory.

The processing may be performed in real time on the mobile communications device. In an exemplary embodiment, the processor processes frame by frame on the mobile communications device in real time. In alternative embodiments, multiple frames are stored and then processed in batches, for example frames from a time period of video recording or from a predetermined number of frames are stored and processed on the phone.Additionally/alternatively, captured video/images are transmitted and processed on the cloud server. A further alternative is that each frame is captured and transmitted to the cloud for processing.

As described above, the facial detection module may include a machine learning (ML) module. The machine learning module is configured to apply one or more deep neural network models. In one example two ML models are used. A first face detection module operates on the image (or frames of a video) for real time facial detection and tracking of the face. A second face mesh module detects the facial features and landmarks of the face and provides locations for face landmarks. The face mesh model may operate on the identified locations to predict and/or approximate surface geometry via regression.

The facial detection module uses the two ML models to identify facial features and landmarks. The identified facial features may be displayed on the screen. These facial features may be used as part of processing the recorded images (or processing each frame of a video recording). The landmarks may be identified and tracked in real time even as the patient may move. ML models use known facial geometries and facial landmarks to predict locations of landmarks in an image.

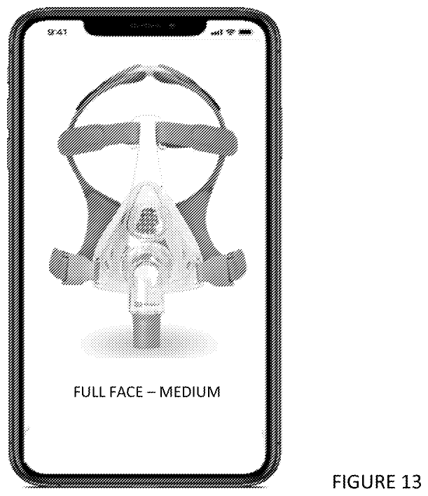

After the dimensions have been calculated at 745, the dimensions are compared to mask data stored in the database to identify a mask suitable for the patient. A mask size that corresponds to the dimensions of the facial features is recommended to the patient at 750. An example of a recommended mask displayed to a patient is shown in FIG. 13. In the example of FIG. 13 the recommended mask is a full face mask, medium size. The application may provide links to purchase options for the patient. For example the application may provide a link that allows purchase of the selected mask and size from a mask retailer or dealer that provides such masks.

Some methods check that the camera is correctly positioned to capture an image of the patient's face. The angle between the camera and the face of the patient is calculated. For example, when the method is implemented on a mobile communications device, for example a phone, the angle may be calculated using sensors within the phone that also comprises the camera. In one example the sensors may comprise one or more accelerometers and one or more gyroscopes.

In some embodiments images are analysed to determine whether attributes of the image meet certain predefined criteria. If the attributes of an image do not meet the predefined criteria, measurements from those images are not used to calculate dimensions of the patient's face. The image may be discarded. This is a filtering step to ignore images in which measurements may be inaccurate, leading to the calculation of incorrect dimensions of the face of the patient. The predefined criteria are predefined filtering criteria. The steps of analysing the image to determine whether the image meets predefined criteria may be performed after the image is processed.

One example of an attribute of an image is the angle of the patient's head with respect to the camera in the image. Further examples of attributes of an image include distance between the camera and the head of the patient, lighting levels, the position of the head within the display and whether all required features are included in the image.

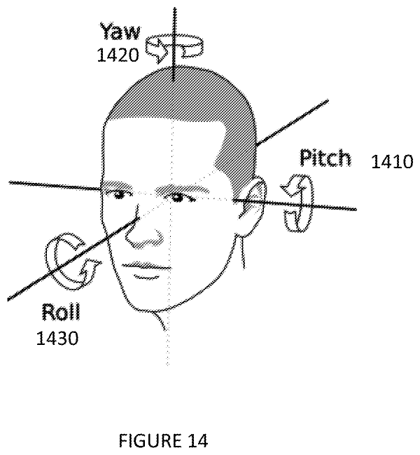

FIG. 14 shows three axes of rotation of the head of a patient. Pitch 1410 is the angle of tilt of the head up and down. Yaw 1420 is the angle of rotation left and right. Roll 1430 is the angle of rotation side to side. The angles of pitch, yaw and roll are measured with respect to the angle of the camera. The accuracy of calculations of dimensions of features within the image may be affected by variations in the angles of pitch, yaw and roll of the image. Images having different angles of pitch, yaw or roll could generate different measurements for certain features and the distance between landmarks of those features may change and landmarks may appear closer together or further apart than they actually are.

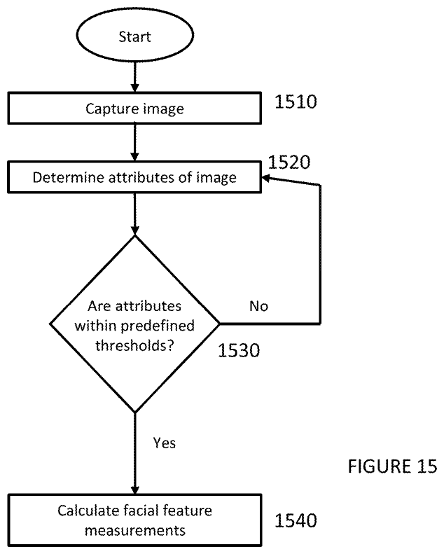

FIG. 15 shows steps that may be implemented by the application to determine whether the attributes of an image meet the predefined criteria. If the attributes of the image meet the predefined criteria, then that image may be used to calculate facial dimensions of the patient. Generally, the steps of FIG. 15 are performed in real time when the image frame is captured at step 720 of FIG. 7.

At 1510, an image is captured by the camera and processed (step 1510 is equivalent to step 720 of FIG. 7). At 1520, the application determines the pitch, yaw and roll angles of the head of the patient within the image and any other required attributes. In exemplary embodiments these attributes are determined in real time.

Various methods may be used to determine the angles of pitch, yaw and roll. In one exemplary method, the application generates a matrix of face geometry. The matrix defines x, y and z values for points on the face in a Euclidean space. The mask sizing application determines pitch, yaw, and roll from relative changes in the x, y, and z Euclidean values as the user's face moves and changes angles. As a user's face moves and changes angles the coordinates of a certain landmark or point can be compared with that landmark's coordinates when the face measures a pitch, yaw, and roll of (0, 0, 0), or a previous angle, or a calibration reference point, to derive the new values of pitch, yaw, and roll at the changed angle. Pitch, yaw, and roll can be measured in +ve and −ve values about various axes that intersect at a common origin point. The x, y, and z points used to measure pitch, yaw, and roll are all measured in relation to the common origin point (0,0,0) that may be located at the Nasion or Pronasale for example.

At 1530, the angles of pitch, yaw and roll are compared against predefined threshold values stored within the memory. These threshold values define tolerance levels for acceptable images. The predefined threshold values may be different for pitch, yaw and roll. In one embodiment the predefined threshold value for pitch angle is 10 degrees in either the +ve or −ve direction. If the pitch angle is greater 10 degrees in either the +ve or −ve direction, then measurements from the image are not used to calculate dimensions of the patient's face.

Predefined threshold values are also applied to yaw and roll. In one example, the predefined thresholds for roll and yaw are greater than 2 degrees in +ve or −ve directions.

Predefined threshold values may vary between embodiments. In one embodiment, the threshold values for pitch is between 10 degrees in the +ve or −ve directions. In exemplary embodiments the threshold value for pitch is 6 degrees in the +ve or −ve directions. Other threshold values may be used in other embodiments. In some embodiments, threshold values may be applied to pitch, yaw and roll. In other embodiments, threshold values may be applied to one or more of pitch, yaw and roll. Typically there is a balance to consider when selecting the tolerance values by selecting values which are sufficiently small to obtain accurate measurement and dimension values, but not so restrictive that it becomes difficult for patients to capture an image which meets the predefined criteria.

If the image meets the predefined threshold criteria at 1530 then the measurements or dimensions of the face of the patient calculated from the image may be used during mask selection at 1540. If the image does not meet the predefined threshold criteria at 1530 then the image is not used in the mask selection process towards a recommendation at Step 750 of FIG. 7.

The filtering steps of determining whether an image meets the predefined criteria may be performed at different stages. The timing of calculating the predefined criteria may be selected based on the processing capabilities of the device, the frame rate, or other factors.

In one embodiment, the dimensions of facial features are calculated regardless of whether the attributes of the image meet the predefined threshold criteria. In such embodiments steps 725 to 745 of FIG. 7 are performed regardless of whether the attributes of the image meet the predefined criteria. The application discards the dimensions calculated from images not meeting the predetermined criteria and these dimensions are not used when selecting a mask for the patient. In other applications, the attributes of the image are calculated and compared against the threshold criteria during image processing immediately after image capture. Images for which the attributes do not meet the required criteria are discarded after Step 720 of FIG. 7 and dimensions are not calculated using these images.

By discarding images in real time, immediately after image capture at Step 720, memory storage and processing load is reduced. Each frame is assessed as it is extracted from a video stream or an image frame buffer. Alternatively, the system may store all or a predetermined number of frames and then assess filtering criteria such as the image attributes described above. By discarding images having attributes which do not meet the predefined criteria, frames that could give the wrong eye width dimension or an inaccurate eye width dimension or give distorted facial features are not considered in the calculation of dimensions.

In some embodiments the application provides the patient with feedback to confirm whether or not the attributes of the image or images being captured by the patient meet the predefined criteria. The feedback may be visual feedback. The feedback may be a visual indicator. The feedback may be text. By providing feedback to the patient, the patient is able to respond to the feedback in real time in order to capture an image which meets the requirements. This can help improve user experience.

The feedback may be haptic feedback. Haptic feedback may include vibrations or a specific vibration pattern to indicate instructions to the user. For example, two short vibrations may mean tilt up and a single short vibration may mean tilt down. Similar haptic feedback can be provided for distance of face to phone, for example three vibrations could be mean move the camera closer to the head and four vibrations could mean move the camera further away from the head.

The feedback may be audio feedback. The audio feedback may provide vocal instructions or sounds to provide instructions to the patient to change the relative orientation or position of the camera with respect to the head. Audio feedback commands are particularly useful to assist patients who are hard of sight.

Some embodiments include a combination of feedback, for example a combination of haptic, visual and audio feedback. Some embodiments may include a combination of haptic and visual feedback, haptic and audio feedback, audio and visual feedback or haptic, visual and audio feedback.

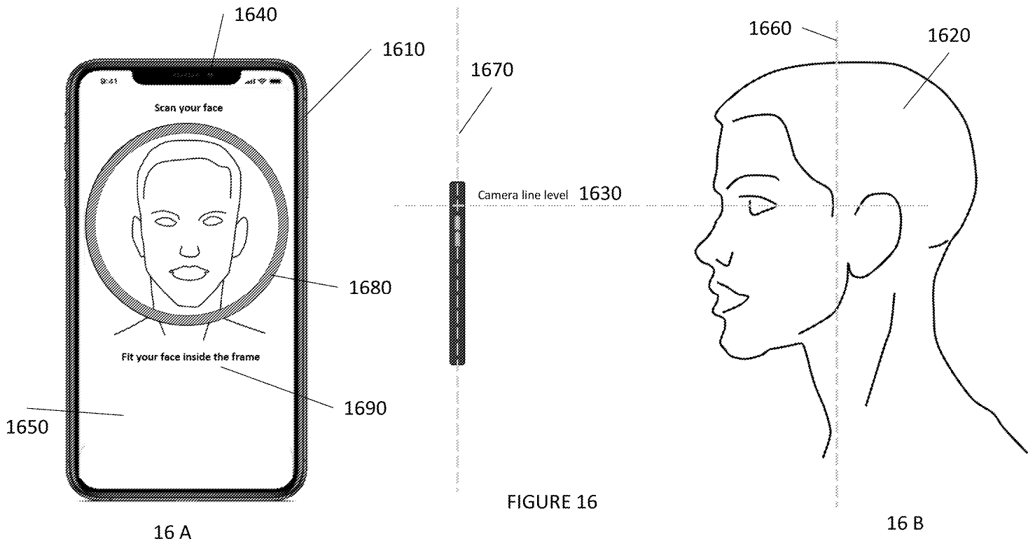

FIG. 16 shows an example of the orientation of a patient's head 1620 with respect to the mobile communications device 1610 during image capture. In the example of FIG. 16 the pitch requirements are met in the image. FIG. 16B is a side view to illustrate the pitch angle of a patient's head with respect to the camera. Similar images could be provided to illustrate yaw and roll angles. The camera 1640 of the mobile communication device is on the front face 1650 of the mobile communications device which includes the display for displaying the image captured by the camera. As discussed above, this arrangement allows the patient to view the image of their face during the image capture process. Camera line level is represented as 1630. The plane of the camera, and so the plane of the image, is represented in FIG. 16B as 1670. The relevant angle of the head of the patient is shown as 1660. In the example of FIG. 16, the head of the patient is directly facing the camera and the angle of the head of the patient relative to the plane 1670 of the camera is approximately zero. This produces a pitch angle of or close to zero. In the example of FIG. 16, the image captured by camera meets the predefined threshold criteria since the pitch angle is within the threshold values. The application provides feedback to the patient confirming that the captured image meets the criteria. This feedback is provided to the patient by presenting a green outline indicator 1680 on the display of mobile communication device 1610. The coloured indicator provides an indication to the user that the user is correctly using the device and that the face is straight. Text feedback 1690 “Fit your face inside the frame” may also be provided on the screen of the mobile communications device.

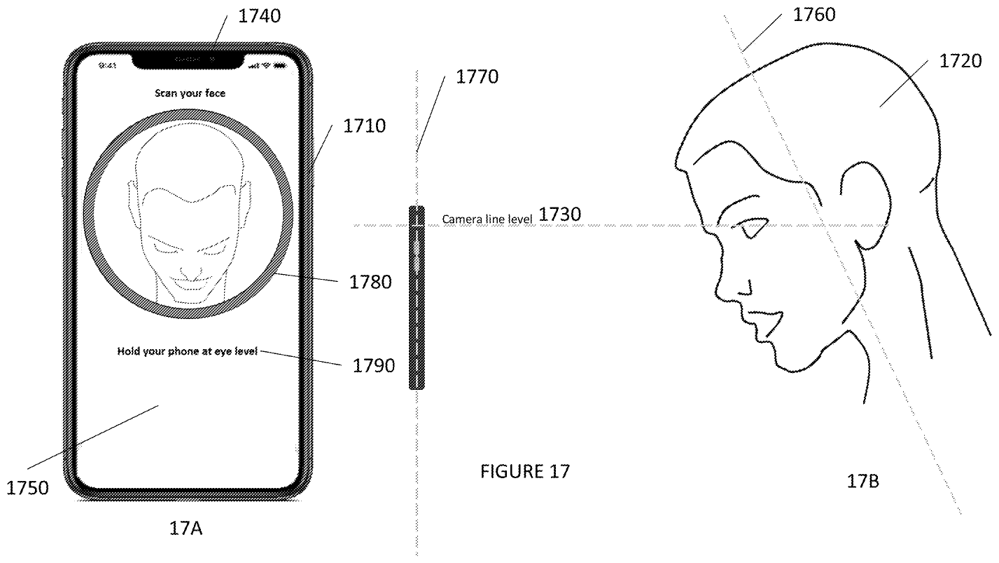

FIG. 17 shows a further example of the orientation of a patient's head 1720 with respect to the mobile communications device 1710 during image capture. In the example of FIG. 17 the pitch requirements are not met in the image. FIG. 17B is a side view to illustrate the pitch angle of a patient's head with respect to the camera. Camera line level is represented as 1730. The plane of the camera, and so the plane of the image, is represented in FIG. 17B as 1770. The angle of the head of the patient is shown as 1760. In the example of FIG. 17, the head of the patient is tilted forwards with respect to the camera plane 1770. This tilt of the head with respect to the camera produces a negative non-zero pitch angle. The head of the patient is not directly facing the camera and an elevated view of the face of the patient appears in the image. In the example of FIG. 17 the pitch angle does not meet the predefined threshold criteria since the pitch angle is outside the threshold values. The application provides feedback to the patient confirming that the captured image does not meet the criteria. This feedback is provided to the patient by presenting a red outline indicator 1780 on the display of mobile communication device 1710. In the example of FIG. 17, further feedback is provided to the patient to help them capture a suitable image in the form of text on the screen of the device. A text feedback instruction 1790 instructs the patient “Hold your phone at eye level”.

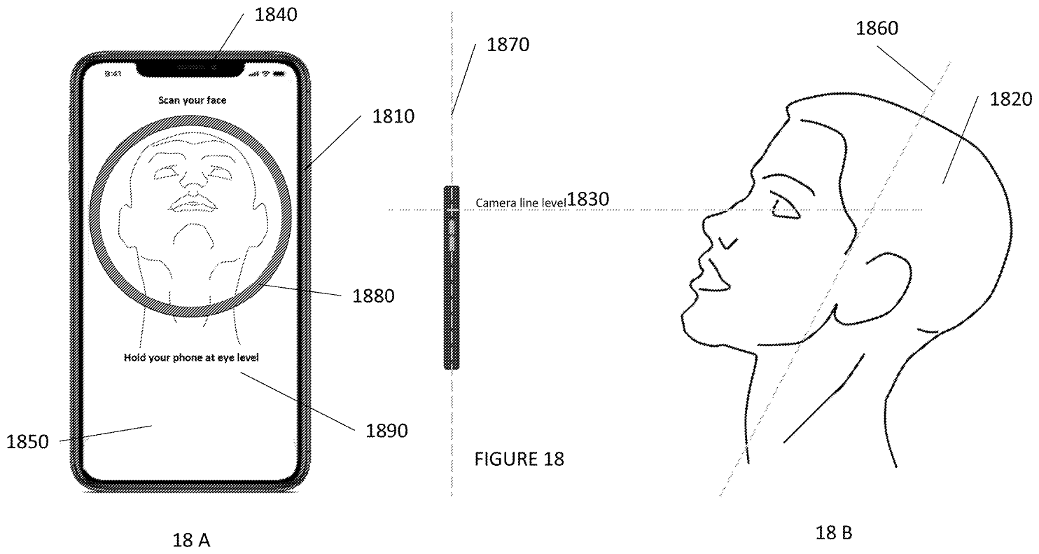

FIG. 18 shows a further example of the orientation of a patient's head 1820 with respect to the mobile communications device 1810 during image capture. In the example of FIG. 18 the pitch requirements are not met in the image. FIG. 18B is a side view to illustrate the pitch angle of a patient's head with respect to the camera. Camera line level is represented as 1830. The plane of the camera, and so the plane of the image, is represented in FIG. 18B as 1870. The angle of the head of the patient is shown as 1860. In the example of FIG. 18, the head of the patient is tilted backwards with respect to the camera plane 1870. This tilt of the head with respect to the camera produces a positive non-zero pitch angle. The head of the patient is not directly facing the camera and an underside view of the face of the patient appears in the image. In the example of FIG. 18 the pitch angle does not meet the predefined threshold criteria since the pitch angle is outside the threshold values. The application provides feedback to the patient confirming that the captured image does not meet the criteria. This feedback is provided to the patient by presenting a red outline indicator 1880 on the display of mobile communication device 1810. In the example of FIG. 18, further feedback is provided to the patient to help them capture a suitable image in the form of text on the screen of the device. A text feedback instruction 1890 instructs the patient “Hold your phone at eye level”.

FIGS. 16, 17 and 18 provide illustrations of various pitch angles of the head of the patient in the image. Similar calculations may be performed for yaw and roll angles and the application may provide similar patient feedback for those angles to reposition the relative positions of the phone and the face if required.

Images are processed in real time during use of the camera by the patient and patient feedback is provided in real time. Thus, the system provides the patient with guidance on using the application to help the patient capture usable images for determining the dimensions of the face. This patient feedback supports non-expert users to capture images which can be used to obtain accurate measurements which can calculate accurate dimensions to be used for mask sizing.

In further embodiments one of the attributes of an image frame is the distance between the face of the patient and the camera. This attribute is used as a filtering criteria to determine whether an image frame is used to calculate a dimension of a facial feature. Preferably the phone is to be held at a predefined distance from the user's face. In one example the set distance is the focal distance or length of the camera. In another example the set distance is based on the reference feature (i.e. eye width). The reference feature, being eye width is allocated a reference dimension such as 28 mm. The distance of a user's face to the camera, and therefore phone, can be calculated using the reference feature dimension and other retrievable measurements such as the focal length of the camera. Such information may be stored in the metadata of a device or an image captured by the device. Further, the measurement of the reference feature as it appears in an image captured by the device can be calculated by the application. This measurement may be in pixels. The following formula may then be used to find the distance of the face from the camera by taking the ratios of the above-mentioned measurements.

$$\frac{Eye width in mm}{D i s tance of face to phone in mm} = \frac{Eye width in pixels}{Focal length ( pixel equivalent )}$$

In one example the predefined distance may be a set distance with a tolerance, for example 30 cm+−5 cm. Alternatively the predefined distance may be defined as a range, for example between 15 cm to 45 cm. Visual feedback is provided to the patient to indicate whether the relative position of the camera and the face of the user are within the predefined distance or range.

As shown in FIGS. 16A, 17A, 18A, visual feedback is provided in the form of an indicator which is displayed on the screen as a circle around the image of the face of the patient. The indicator (circle around the face) is a first colour (e.g. red) when the phone is not held at the predefined distance or does not meet other required attributes. If the phone is held at the set distance, then the indicator (circle) is green to indicate that the predefined attributes are met. This is advantageous because it provides a user an easy to understand and visual indicator in order to correctly position the mobile communications device. Further the visual indicator is advantageous because it provides real time feedback to correctly position their head and mobile communications device. Optionally real time audio feedback and/or real time haptic feedback can also be provided. Audio feedback and haptic feedback can be optionally provided in combination with the visual feedback presented on the screen of the mobile communications device.

Further exemplary embodiments collect subjective data from the patient in addition to the image data of the face of the patient. Embodiments include questions which are presented to the patient. In an example embodiment the questions are stored in the memory. In exemplary embodiments, the questions are presented on the display of the mobile communications device. The patient is prompted to respond to the question by providing a response. In an example embodiment, the response is received through user input device 425. The question may be a YES/NO question or a question having predefined response options which are presented to the patient.

The application presents the questions to the patient as part of the mask selection process. The questions are presented in addition to the image capture process described above. The questions are another part of the process for data collection or data processing during mask selection.

The patient responses in the form of the subjective data described above are used in the selection of a mask category for a patient. The responses from the patient are used to help the application to identify which masks are most suitable for the patient. The patient response may be used in combination with the dimension data calculated from the image of the patient's face to recommend a mask to the patient.

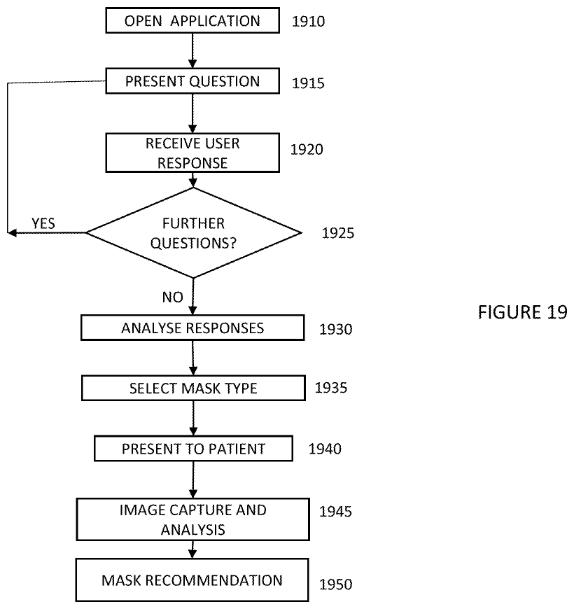

An embodiment including patient questions is now described with respect to FIG. 19. In the following embodiment, the questions are presented to a patient on activation of the mask sizing application. The questions are presented and responses received before the application initiates the camera for the image capture process.

The questions are provided to support the mask selection software application in recommending an appropriate mask or an appropriate group of masks or a mask category for the patient. In the following example, the questions are presented to a patient to select a mask type or mask category suitable for the patient. Mask categories include full face mask, nasal mask, sub-nasal masks, under nose masks. As discussed above, each mask category fits differently onto the face of the patient and may engage with different features of the patient's face.

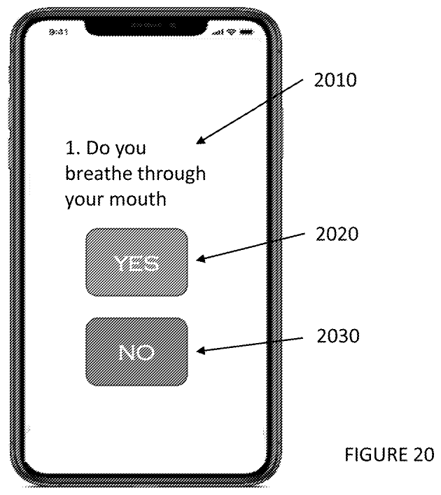

At 1910 the mask selection software application is accessed by a patient on a mobile communications device. At 1915, a question is presented to the patient. In an example embodiment the questions are presented on the screen of the mobile communications device. The questions may be presented individually or collectively. FIG. 20 is an illustration of a question being presented on the screen of a mobile communications device. The question is presented as text 2010 and asks the patient “Do you breathe through your mouth?”. The user is presented with response options YES 2020 or NO 2030. Preferably the display is a touchscreen display and the patient can provide a response by touching the appropriate response text on the display. The response is received by the application at 1920.

In other embodiments, audible questions are presented to the patient. Voice recognition software of the phone may be used to receive a vocal response from the patient. One example of suitable software is Apple's Siri application or Android's Voice Access application. The application may be used to present the question to the patient. Patient responses may be provided via the touchscreen via a virtual button or via an audible manner by the user in which the patient can speak their response.

Multiple questions may be presented sequentially. In an example embodiment, all questions are YES/NO questions, but in some embodiments additional predefined responses may be presented, or the patient may be able to provide an independent open text response.

Different question sets may be provided to different patients. In one example the application presents an initial question at 1915 to determine whether the patient has previously used a Positive Airway Pressure (PAP) device. Different question sets or question sequences are presented to the patient depending on whether the patient has previously used a PAP device or not.

At 1915 the patient is asked the question:

### HAVE YOU USED A PAP DEVICE OR MASK BEFORE?

The user is presented with response options YES and NO. User response is received at 1920.

At 1925, the application identifies the patient response and determines which question to ask next. The following sequences of questions are examples of sequences of questions which may be presented to the patient depending on whether they answer YES or NO to the question HAVE YOU USED A PAP DEVICE OR MASK BEFORE? The questions may be presented sequentially, displaying a single question at a time and waiting for the patient response before displaying the next question to the patient. Alternatively, the questions may be displayed concurrently or in groups.

In the exemplary embodiment, if the patient answers NO to the question HAVE YOU USED A PAP DEVICE BEFORE?, the application presents the following questions to the patient:

| QUESTION | ANSWER OPTIONS |
|---|---|
| ARE YOU A RESTLESS SLEEPER? | YES/NO |
| DO YOU WEAR GLASSES IN BED BEFORE | YES/NO |
| SLEEP? | |
| DO YOU HAVE SENSITIVE NOSTRILS? | YES/NO |
| DO YOU STRUGGLE TO HANDLE THINGS/ | YES/NO |
| HAVE ANY DEXTERITY ISSUES? | |
| WHAT IS YOUR PREFERRED SLEEPING | BACK/SIDE/STOMACH |
| POSITION? | |
| DO YOU GET CLAUSTROPHOBIC OR | YES/NO |
| ANXIOUS? | |

In the exemplary embodiment if the patient answers YES to the question HAVE YOU USED A PAP DEVICE BEFORE?, the application presents a different set of questions to the patient:

| QUESTION | ANSWER OPTIONS |
|---|---|
| WHAT CATEGORY OF MASK HAVE YOU | ORONASAL (FULL FACE)/NASAL/SUB |
| USED BEFORE/ARE CURRENTLY | NASAL |
| USING? | |
| WHAT DO YOU LIKE/DID YOU LIKE | SMALL AND COMPACT/COMFORTABLE/ |
| ABOUT YOUR MASK? | SIMPLE TO USE/COLOUR SHAPE/ |
| | NOTHING |
| WHAT DO YOU DISLIKE/DID YOU | BULKY/UNCOMORTABLE/LEAK ISSUES/ |
| DISLIKE ABOUT YOUR MASK? | HARD TO USE AND FIT/NOTHING |
| ARE YOU A RESTLESS SLEEPER | YES/NO |
| DO YOU WEAR GLASES IN BED BEFORE | YES/NO |
| SLEEP? | |
| DO YOU HAVE SENSITIVE NOSTRILS? | YES/NO |
| DO YOU STRUGGLE TO HANDLE | YES/NO |
| THINGS/HAVE ANY DEXTERITY | |
| ISSUES? | |
| WHAT IS YOUR PREFERRED SLEEPING | BACK/SIDE/STOMACH |
| POSITION? | |
| DO YOU GET CLAUSTROPHOBIC OR | YES/NO |
| ANXIOUS? | |

The questions listed above are a combination of YES/NO questions and multiple choice questions. Questions may also include an option to answer “I don't know”. This allows a more suitable score to be calculated for patients who do not know an answer to a question and prevents the patient guessing a YES or NO answer. Further embodiments may include different questions. Further embodiments include options for a patient to provide a free text response. Further examples do not have an initial question that determines the presentation of subsequent questions. Further examples have questions update as the user progresses through the questionnaire in the from of questions being skipped or changing the content of questions or further questions being added.

The sequence of questions may be predefined and fixed. In further embodiments the sequence of questions may be dependent on the responses provided by patients and the application determines which question to present next based on previous responses.

On receipt of the response by the application at 1920, the application determines whether any further questions are required at 1925. If yes, a further question is presented to the patient at 1915. If not, the patient responses are analysed at 1930. Optionally, the application may not present a single question if the user (e.g. patient) answers YES to the question HAVE YOU USED A PAP DEVICE BEFORE? If the user answer's YES, then the application may present a question such as PLEASE SELECT THE MASK CATEGORY THAT YOU USE/HAVE USED BEFORE. The application may then present the available mask categories e.g. Full Face, Nasal, Under Nose etc.

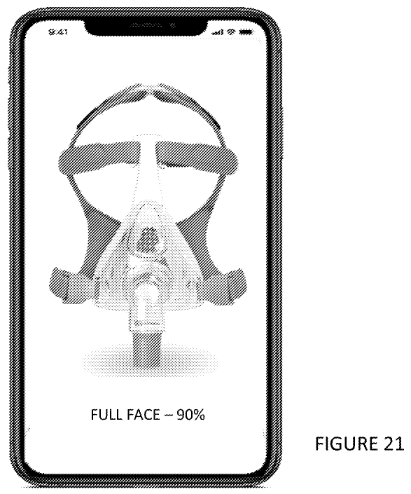

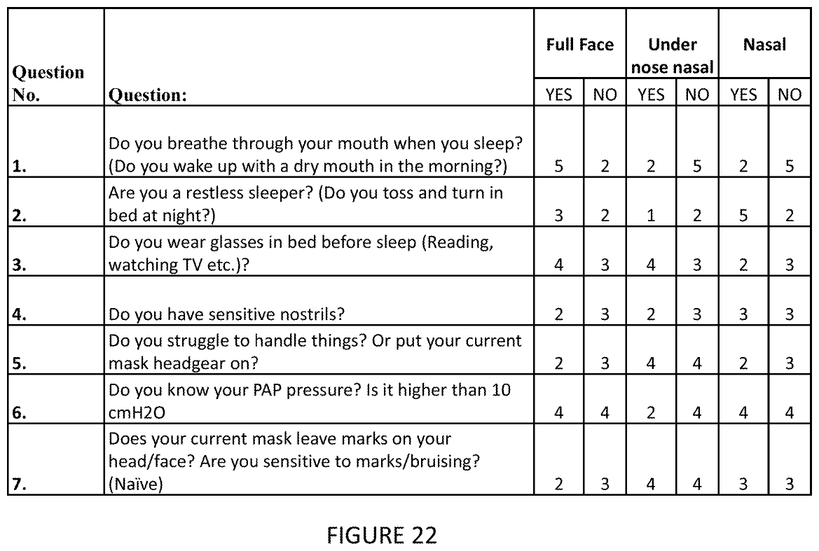

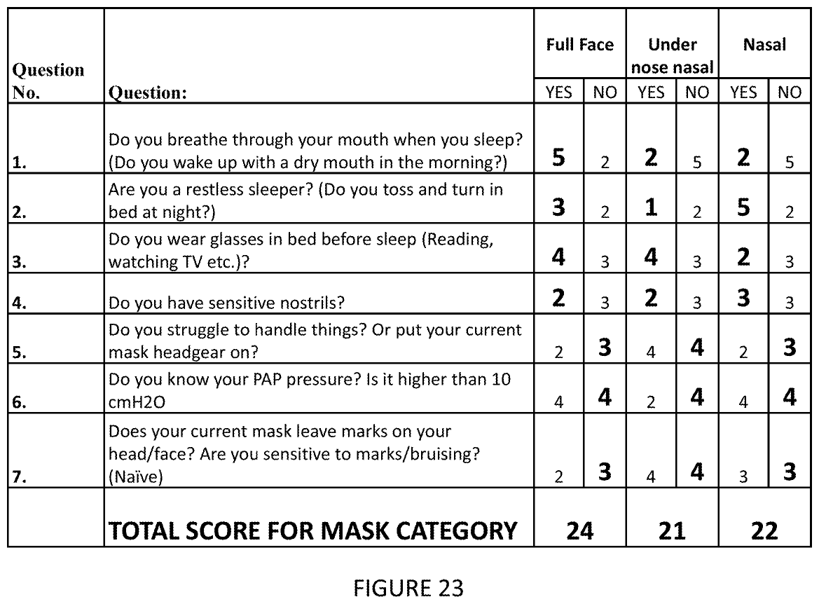

In one example, described in more detail below with reference to FIGS. 22 and 23 each of the responses received by the application is provided a score and weighted. The overall score for the patient is calculated. Mask categories are provided with specific scores and a mask category recommendation is generated at 1935. In other embodiments a list of two or more mask categories may be recommended, for example in order of suitability. The mask category recommendation may be displayed on the mobile communications device at 1940. Further information may be displayed with the mask recommendation. Examples of further information include an image of the mask, information about the mask, for example mask category, or relevance of the mask. FIG. 21 provides an example of a display identifying that a full face mask is recommended to the patient. The display identifies that the full face mask provides a 90% match based on the answers provided by the patient.

FIG. 22 illustrates an example of a scoring table associated with a series of questions presented to a patient. The questionnaire includes seven questions presented to the patient. In the example of FIG. 22, each question has a YES/NO answer. The patient responses are collected and mapped against three different mask categories, namely FULL FACE, UNDER NOSE NASAL, NASAL. Additional categories and associated mapping of answers may also be included.

The table shown in FIG. 22 is used to calculate suitability scores for each mask for a specific patient, based on the answers to the questions of that specific patient. This step is performed at Step 1930 of FIG. 19. As each mask has different characteristics, each question may have a different relevance/weighting for different masks. The weighting Is represented by different scores allocated to the YES/NO responses for the different masks, as shown in FIG. 22. For example, nasal mask provides a high score of 5 for a ‘no’ answer to the question asking if patient breathes through their mouth, since these masks are suitable for patients who breathe through their nose. The specific scores are generated based on various clinical studies and other research and can be tweaked and recalibrated in the future.

Some questions might be neutral for a specific mask, in which case the score given for that question is the same regardless of the answer the patient gives indicating that that question has little importance/relevance for that specific mask. An example question is Question 5, “Do you struggle to handle things? Or put your current mask headgear on?”. The patient scores a “4” regardless of whether the input answer is YES or NO for the under the nose category because this question has little relevance for that specific category.

An example of the patient responses to the questions of FIG. 22 are now described with reference to FIG. 23 to illustrate how the mask selection software application uses the patient responses to select a mask category for the patient. The patient responses are shown in the following table:

| QUESTION | ANSWER |
|---|---|
| DO YOU BREATHE THROUGH YOUR MOUTH WHEN YOU | YES |
| SLEEP (DO YOU WAKE UP WITH A DRY MOUTH IN THE | |
| MORNING)? | |
| ARE YOU A RESTLESS SLEEPER (DO YOU TOSS AND | YES |
| TURN AT NIGHT)? | |
| DO YOU WEAR GLASSES IN BED BEFORE SLEEP | YES |
| (READING, WATCHING TV, ETC)? | |
| DO YOU HAVE SENSITIVE NOSTRILS? | YES |
| DO YOU STRUGGLE TO HANDLE THINGS? OR PUT | NO |
| YOUR HEADGEAR ON? | |
| DO YOU KNOW YOUR PAP PRESSURE? IS IT HIGHER | NO |
| THAN 10 cmH20? | |
| DOES YOUR CURRENT MASK LEAVE MARKS ON YOUR | NO |
| HEAD/FACE? ARE YOU SENSITIVE TO | |
| MARKS/BRUISING? | |

The answer to each question generates a score for each mask category which depends on the suitability of that mask to the response provided by the patient. For example, question 1: Do you breathe through your mouth when you sleep? (Do you wake up with a dry mouth in the morning?). The patient input answer YES. The answer YES scores 5 in the Full Mask category. This is a high score indicating that the full face mask category is suitable for patients who breathe through their mouths. The answer YES only scores 2 in the under nose nasal and nasal mask categories, indicating that these masks are less suitable for patients who breathe through their mouths.

In the example, question 6: Do you know your PAP pressure? Is it higher than 10 cmH2O?, the patient has answered “NO”. This answer scores 4 in each of the mask categories. This indicates that none of the masks are more suitable than the others for a patient who does not know their PAP pressure. This is an example of a neutral response.

The mask scores for the patient based on the responses provided are calculated for each category of mask. In the example shown in FIG. 23, the highest scoring mask category for the patient is Full Face. The lowest scoring mask category is Under nose nasal. These scores indicate that the most suitable mask category for the patient is a full face mask. As discussed above, after a mask category is determined for a patient, the mask category may be displayed to the patient at 1940. FIG. 21 shows an example of a display screen presenting a mask category to the patient.

In an exemplary embodiment the questionnaire is presented to the patient in a first stage of the mask selection process. After the responses have been received by the application, the application enters a second stage of the mask selection process at 1945, to capture an image of the patient's face and calculate dimensions of the patient's face. The second stage of the mask selection process follows many of the steps described above with respect to FIG. 7. Typically, the first stage of the mask selection process of presenting the questions to the patient, is concerned with selecting the most suitable mask category. The second stage of the mask selection process is concerned with sizing the mask for the patient and selecting the most appropriate size mask in the suitable mask category.

On completion of the image capture and analysis at Step 1945 of FIG. 19, the application selects and recommends a mask to the patient at 1950 using the questionnaire data and the image data.

Different mask categories contact the face at different points of the face, as shown in FIGS. 2 and 3 and described above. Consequently, different facial dimensions are relevant when fitting masks of different categories. In some cases, some facial dimensions may be more dominant than others in fitting, or some dimensions may not be required.

The patient responses are used to identify which mask categories will be included in mask sizing. The following paragraphs provide examples of facial dimensions that may be relevant for different mask categories. After determining the most suitable mask category for a patient, example embodiments of the application calculate dimensions of facial features relevant for the determined mask category and use these dimensions to select the size of mask within the determined category.

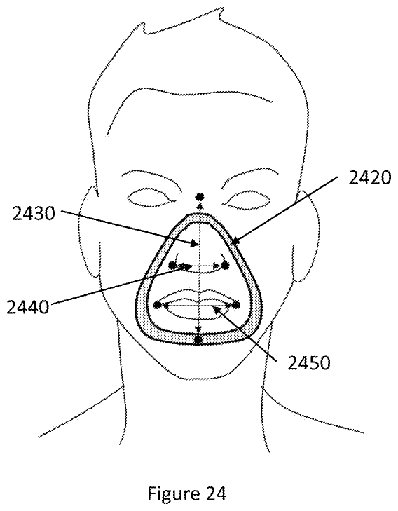

FIG. 24 illustrates the seal 2420 between the mask and the face for a full face mask. For a full face mask, example relevant feature dimensions for sizing are shown in FIG. 24. A first relevant dimension is the dimension 2430 from the nasal bridge to the lower lip. Referring to FIG. 10, this is the dimension from landmark (d) nasion to landmark (m) sublabial. A second relevant dimension is the width of the mouth 2450. Referring to FIG. 10, this is the dimension between landmark (k) left labial commissure and landmark (l) right labial commissure. A third relevant dimension is the width of the nose 2440. Referring to FIG. 10, this is the dimension between landmark (h) left alare and landmark (i) right alare.

Referring now to FIG. 19, if the application determines that a patient requires a full face mask at 1935, based on patient responses to the patient questionnaire at 1920, during image analysis at 1945, the application retrieves the coordinates of the six example landmarks relevant to sizing a full face mask, namely: (d) nasion; (m) sublabial; (k) left labial commissure; (l) right labial commissure; (h) left alare and (i) right alare. The dimensions of the features defined by the landmarks, namely: nasal bridge to lower lip; width of the mouth; and, width of the nose, are calculated. The dimensions are then compared with the mask sizing data including dimensions or thresholds to determine which size mask is suitable for the patient. The mask sizing data may be stored in memory 420 of mobile communications device 400. By storing the mask sizing data on the mobile communications device the application is able to recommend a mask to the patient without requiring a network connection.

In embodiments, the facial detection module determines the coordinates for all facial landmarks in the image. The application identifies the landmarks relevant to the specific mask category and retrieves those coordinates to calculate the measurements of the relevant facial features in the image and the dimensions of those relevant facial features.

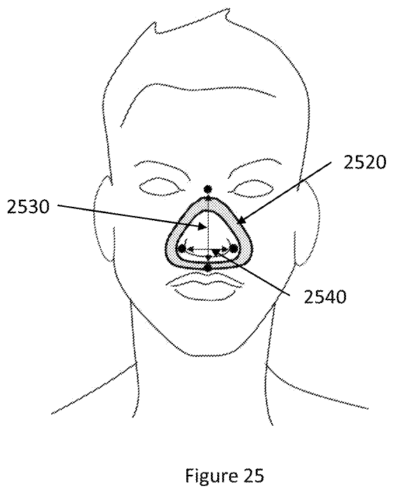

The sizing process is now described for a nasal face mask with reference to FIG. 25. For a nasal face mask, the relevant facial features are nose height 2530 and nose width 2540. The facial feature of nose height is defined between facial landmark (d) nasion and landmark (j) subnasale. The facial feature of nose width is defined between the left and right alar lobule (landmarks h and i of FIG. 10). Referring again to FIG. 19, when the application determines that a patient requires a nasal face mask at 1935 based on responses to the patient questionnaire at 1920, during image analysis, the application retrieves the coordinates of the four example landmarks relevant to sizing a nasal face mask, namely: (d) nasion; (j) subnasale; left alar lobule (h) and (i) right alar lobule. The dimensions of the features defined by the landmarks, namely: nose height and nose width are then compared with the mask sizing data including dimensions or thresholds to determine which size mask of nasal face mask is suitable for the patient.

The table below provides example sizing data for nasal face masks. A recommended mask size is provided for various nose heights and nose widths. In an exemplary embodiment, the data is stored as a look up table in memory 420 and the application references the sizing data to select a mask size for the patient.

| | | DIMENSION OF NOSE HEIGHT | | |
|---|---|---|---|---|
| | | <4.4 cm | 4.4 cm-5.2 cm | >5.2 cm |
| Dimension of | <3.7 cm | S | M | L |
| Nose Width | 3.7 cm-4.1 cm | M | M | L |
| | >4.1 cm | M | L | L |

The mask sizing data in the table is for sizing nasal face masks. The look up table provides a known result for the various possible combinations of the dimensions of the relevant features. For example, for nasal masks if the patient's nose height is calculated to be between 4.4-5.2 cm and nose width is calculated to be greater than 4.1 cm, then the most suitable size is a large (L).

Similar look up tables are provided for each mask category. For example, to size a full face mask with n relevant dimensions, an n-D lookup table would be used, that is a lookup table or function with n number of input parameters produces known results based on the various possible combinations of the input parameters and their different ranges. Different masks may have different sizing charts, lookup tables, or sizing functions. The look up tables are stored in memory.

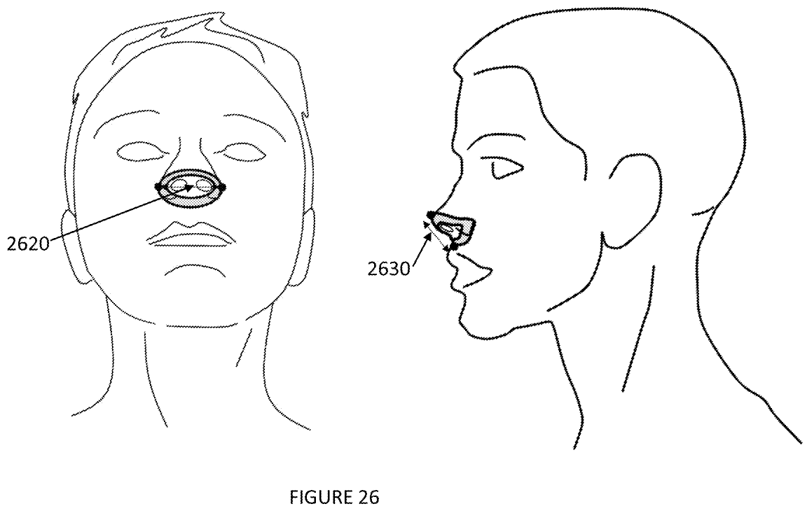

The sizing process is now described for under nose nasal masks, with reference to FIG. 26. For under nose nasal masks, the relevant facial features are nose width 2620 and the nasal length 2630 (i.e. nasal depth). This is because the seal sits under the nose and wraps around under the nose.

Nose width is defined as the dimension between the left alar lobule (feature h in FIG. 10) and the right alar lobule (feature i in FIG. 10). Nasal length is determined for example based on the distance of the pronasal tip (feature g in FIG. 10) to the subnasale (feature j in FIG. 10). Referring again to FIG. 19, when the application determines that a patient requires an under nose nasal mask at 1935 based on responses to the patient questionnaire at 1920, during image analysis, the application retrieves the coordinates of the four example landmarks relevant to sizing an under nose nasal face mask, namely: left alar lobule (h); (i) right alar lobule; pronasal tip (g); and, subnasale (j). The dimensions of the features defined by the landmarks, namely: nasal length and nose height are compared with mask sizing data including dimensions or thresholds to determine which size mask of under nose nasal mask is suitable for the patient. The dimensions may be calculated using all three (x,y,z) coordinates for the four landmarks, or just using y and z.

As described above, in exemplary embodiments, the selection of the mask category for a patient from the responses to the questionnaire is used to determine which dimensions may be required for mask sizing. The questionnaire is presented first and the patient responses are used to determine the category of mask. Once the category is identified, the specific landmarks that are required for that mask category are identified in the application. All landmarks may be gathered, but the calculation of distance between specific landmarks are done by the application based on the mask category identified.

Other methods may be used for determining a mask category for a patient. For example, the application may be preconfigured with a particular mask category for a patient or the application may rely on a patient selecting a mask category.

In the embodiments described above, the application and various databases have been stored locally on the mobile communications device. Additionally, all processing during mask selection is performed on the mobile communications device. This arrangement avoids the need for any network connections during a mask selection process. Local processing and data retrieval may also reduce the time taken to run the mask selection process. One advantage is that questions and images can be processed locally and only the calculated mask size needs to be transmitted, for example when ordering a product. This reduces the data sent and reduces data costs.

However, further embodiments execute the mask sizing application using a distributed data storage and processing architecture. In such embodiments, databases, for example the mask sizing database, or questionnaire database, may be located remotely from the mobile communications device and accessed via a communication network during execution of the mask selection application. Processing, for example facial landmark identification may be performed in remote servers and the mobile communications device may send captured images across the communications network for processing. In other examples, processing of questionnaire responses may be done remotely. Such embodiments leverage external processing capabilities and data storage facilities.

In the embodiments described above the application has been executed on a mobile communications device. In further embodiments the application, or parts of the application, may be executed on a respiratory therapy device.

The examples described provide an automated manner of recommending a mask category and a mask size in the specific category of mask that is selected for the patient. Embodiments are configured to enable a non-professional user using non-professional equipment to capture data to enable the selection of a suitable mask for use with a respiratory therapy device. Sizing determination can take place using a single camera which allows the application to be executed on smartphones or other mobile communication devices. Embodiments do not require use of any other phone functions/sensors e.g. accelerometers.

Embodiments provide an application which allows for remote mask selection and sizing. This allows for remote patient set up and reduces the need for the patient to come into a specialist office for mask fitting and set up. The application can also provide general mask information and provide instructions regarding user instructions, cleaning instructions and troubleshooting as additional information.

The application uses the palpebral fissure width as a reference measurement within the image of the face of the patient. The palpebral fissure is detectable in a facial image using facial feature detection software and is less likely to be obscured by the eye lid of the patient compared with features of the eye, for example the iris or pupil. The greater width of the eye, compared with smaller facial features or eye features like the iris, enables the application to capture accurate measurements even when the patient does not hold their head still or the device being used is not able to capture higher resolution images. Use of the palpebral fissure as a reference measurement also allows the application to measure a single eye width or measurement of two eye widths to be measured and averaged. The corners of the eye can also be detected from the contrast between the whites of the eye and the skin.

Embodiments account for tilt of the patient's head and filters out measurement that may cause errors due to excessive tilt (i.e. pitch). Similar filtering can be used for roll and yaw. The described embodiments are also advantageous because the tilt does not use the inertial measurement unit (e.g. an accelerometer or gyroscope) of the mobile communications device which can reduce the processing load and time on the processor of the mobile communications device. This also means that less sophisticated devices which might not have inertial measurement units can still be used to implement the described examples.

The sizing measurements can be performed even when the phone distance from the face varies. There is a preferred distance to ensure that the facial features of interest are captured at a high enough resolution to obtain accurate dimensions. There is a visual guide that helps the user navigate and use the sizing app. Sizing can be performed in many different environments e.g. outdoor light, indoor light. Sizing can be performed regardless of user orientation i.e. user can be lying down or sitting or standing. This provides a more robust sizing app to size patient interfaces.

Example embodiments are configured to capture images from a single image only and the patient is not required to take profile images or multiple images from different angles.

Example embodiments provide real time processing of images/video frames. This reduces processing loads and doesn't require large caching/memory requirements. Exemplary embodiments do not require large memory or caching, frames/images are not stored but processed and discarded as received.

The examples above describe ‘selecting’. In example embodiments the selection involves identifying a mask.

It is to be understood that, if any prior art publication is referred to herein, such reference does not constitute an admission that the publication forms a part of the common general knowledge in the art, in Australia or any other country.

In the claims which follow and in the preceding description, except where the context requires otherwise due to express language or necessary implication, the word “comprise” or variations such as “comprises” or “comprising” is used in an inclusive sense, namely, to specify the presence of the stated features but not to preclude the presence or addition of further features in various embodiments of the invention.

It is to be understood that the aforegoing description refers merely to exemplary embodiments of the invention, and that variations and modifications will be possible thereto without departing from the spirit and scope of the invention, the ambit of which is to be determined from the following claims.

1. A method for selecting a mask for a patient for use with a respiratory therapy device, the mask suitable to deliver respiratory therapy to the patient, comprising the steps of:
receiving data representing at least one digital image of a face of a patient;
identifying a predefined reference facial feature appearing in the at least one digital image, the predefined reference facial feature being an eye of the patient;
determining a measurement for the eye of the patient within the at least one digital image, wherein the measurement for the eye of the patient is one of: an eye width and an eye height;
allocating a predefined dimension to the measurement, and
determining a scaling factor for the at least one digital image, the scaling factor being a ratio between the measurement and the predefined dimension;
identifying a further facial feature in the at least one digital image;
determining a measurement of the further facial feature in the at least one digital image; and calculating a calculated dimension of the further facial feature using the scaling factor and the measurement of the further facial feature; and,
comparing the calculated dimension of the further facial feature with mask sizing data associated with patient masks and selecting a mask for the patient in dependence on the comparison.

2. (canceled)

3. (canceled)

4. A method according to claim 1, wherein the step of identifying an eye of the patient in the at least one digital image is performed by identifying at least two predefined facial landmarks, being anthropometric features of the face of the patient, in the at least one digital image associated with the eye.

5. A method according to claim 4, wherein the at least two predefined facial landmarks in the at least one digital image are corners of the eye or a medial canthus and a lateral canthus.

6. (canceled)

7. (canceled)

8. A method according to claim 1, wherein the further facial feature is identified by identifying at least two facial landmarks associated with the further facial feature.

9. (canceled)

10. A method according to claim 1, wherein the step of determining a measurement of a facial feature is performed by calculating a number of pixels of the at least one digital image between at least two facial landmarks in the at least one digital image associated with the facial feature.

11. A method according to claim 1, wherein the step of determining the measurement for the eye of the patient within the at least one digital image is performed by identifying two eyes of the patient within the at least one digital image and calculating a measurement for each eye and calculating an average measurement for the two eyes.

12. (canceled)

13. A method according to claim 1, comprising the further steps of:
determining at least one attribute of the at least one digital image;
comparing the at least one attribute with predefined attribute criteria; and determining whether the at least one attribute meets the predefined attribute criteria;
wherein at least one of the steps of;
identifying an eye of the patient;
determining a measurement for the eye of the patient;
allocating a predefined dimension to the measurement;
determining a scaling factor;
identifying a further facial feature;
determining a measurement of the further facial feature;
calculating a calculated dimension of the further facial feature; and
selecting a mask for the patient
is performed in dependence on the at least one attribute meeting the predefined attribute criteria.

14. A method according to claim 13, wherein the at least one attribute comprises at least one of:
an angle of the face of the patient within the at least one digital image, the angle being at least one of a pitch angle, a yaw angle or a roll angle;
a focal length of the at least one digital image;
depth of the face of the patient in the at least one digital image; and
at least one predefined landmark being identified in the at least one digital image.

15. A method according to claim 13, wherein the at least one attribute is a pitch angle, the predefined attribute criteria being an angle between 0 to +−6 degrees with respect to a plane of the at least one digital image.

16. A method according to claim 13, comprising the further step of providing feedback relating to whether the at least one attribute meets the predefined attribute criteria.

17. A method according to claim 1, wherein the step of calculating the calculated dimension of the further facial feature is performed for multiple images of the at least one digital image, to produce multiple calculated dimensions, the method comprising the further step of calculating an average dimension of the further facial feature across at least a predetermined number of the multiple images; and using the average dimension to compare with the mask sizing data.

18. (canceled)

19. A method according to claim 17 comprising the steps of:
determining at least one attribute of the multiple images;
comparing the at least one attribute with predefined attribute criteria; and determining whether the at least one attribute meets the predefined attribute criteria;
wherein the average dimension is calculated across the multiple images which meet the predefined attribute criteria.

20. A method according to claim 1 comprising the further step of determining a determined mask category for the patient.

21. A method according to claim 1 comprising the further steps of:
presenting at least one patient question to the patient;
receiving at least one patient response to the at least one patient question; and
determining a determined mask category for the patient in dependence on the at least one patient response.

22. A method according to claim 20, wherein the further facial feature is selected from a plurality of facial features in dependence on the determined mask category, wherein different mask categories have different relationships between mask sizing data and dimensions of facial features, and wherein the mask sizing data for the determined mask category includes data relating to the selected further facial feature of the plurality of facial features.

23. (canceled)

24. (canceled)

25. (canceled)

26. A system for selecting a mask for a patient for use with a respiratory therapy device, the mask suitable to deliver respiratory therapy to the patient, the system comprising:
a processor configured to:
receive data representing at least one digital image of a face of a patient;
identify a predefined reference facial feature appearing in the at least one digital image, the predefined reference facial feature being an eye of the patient;
determine a measurement for the eye of the patient within the at least one digital image, wherein the measurement for the eye of the patient is one of: an eye width and an eye height;
allocate a predefined dimension to the measurement, and
determine a scaling factor for the at least one digital image, the scaling factor being a ratio between the measurement and the predefined dimension;
identify a further facial feature in the at least one digital image;
determine a measurement of the further facial feature in the at least one digital image; and calculate a calculated dimension of the further facial feature using the scaling factor and the measurement of the further facial feature; and, a memory for storing mask sizing data associated with patient masks;
the processor further configured to:
compare the calculated dimension of the further facial feature with the stored mask sizing data associated with patient masks and select a mask for the patient in dependence on the comparison.

27-84. (canceled)

85. The system according to claim 26, wherein the system comprises a mobile communications device, the mobile communications device further comprising:
an image capture device for capturing digital image data; and
a user interface to display data related to the at least one selected mask.
